(19) United States
(12) Patent Application Publication
(10) Pub. No.: US 2013/0039872 A1
(43) Pub. Date: Feb. 14, 2013

(54) ESTER PREPARATION WITH AFTER-TREATMENT

(71) Applicants: Peter Daute, Beverstedt (DE); Wilhelm Reiners, Loxstedt (DE); Martin Schäfer, Stubben (DE); Udo Frerichs, Loxstedt (DE); Hinrich Hildebrandt, Armstorf (DE); Joern Ellerbrake, Bremerhaven (DE)

(72) Inventors: Peter Daute, Beverstedt (DE); Wilhelm Reiners, Loxstedt (DE); Martin Schäfer, Stubben (DE); Udo Frerichs, Loxstedt (DE); Hinrich Hildebrandt, Armstorf (DE); Joern Ellerbrake, Bremerhaven (DE)

(21) Appl. No.: 13/519,981

(22) PCT Filed: Dec. 30, 2010
(86) PCT No.: PCT/EP2010/007975

§ 371 Date: Oct. 22, 2012

(30) Foreign Application Priority Data
Dec. 30, 2009 (DE) 10 2009 060 851.6

(51) Int. Cl. C07C 67/08; C07C 69/602; C07C 69/50; C07C 69/533; C07C 67/56; B65B 1/04; C10M 169/04; C12N 9/00; C08K 5/11; C09K 8/18; C09K 3/00; B29C 51/10; A61K 8/37; B01J 8/00

(52) U.S. Cl. 424/70.1; 554/223; 560/204; 514/547; 514/549; 560/190; 554/191; 560/191; 422/187; 508/459; 435/183; 524/310; 507/139; 516/133; 264/500; 53/473

(57) ABSTRACT

The present invention relates to a process for the preparation of an ester in a reactor, a device, a process for the preparation of a thermoplastic composition comprising the ester prepared according to the invention, a process for the production of a shaped article comprising the ester according to the invention or the thermoplastic composition according to the invention, a process for the production of a packed product, a process for the production of an at least partly coated object, and uses of the esters according to the invention as an additive in various compositions.

The present invention relates to a process for the preparation of an ester in a reactor, a device, a process for the preparation of a thermoplastic composition comprising the ester prepared according to the invention, a process for the production of a shaped article comprising the ester according to the invention or the thermoplastic composition according to the invention, a process for the production of a packed product, a process for the production of an at least partly coated object, and uses of the esters according to the invention as an additive in various compositions.

Esters, in particular those based on aliphatic carboxylic acids and alcohols, are employed successfully in a large number of uses. In the awareness that raw materials from fossil deposits are becoming scarcer, new sources of raw materials are being sought. Oils from animal or plant renewable raw materials which are broken down to fatty acids e.g. by ozonolysis and refunctionalized or derivatized in further steps appear to be particularly promising.

Ester preparation is an industrially important derivatization, for which various processes are known. These can be categorized in various ways. One possibility is classification into low temperature and high temperature processes. In this context, generally, low temperature processes are often more gentle, i.e. generate fewer side reactions and decomposition or oxidation products, and high temperature processes are characterized by higher rates of reaction.

In conventional low temperature processes, as a general rule proton acids or sulphonic acid derivatives are added as catalysts. In the case of proton acids in particular, such as sulphuric acid or phosphoric acid, by-products, in particular unsaturated compounds, are formed in a considerable proportion. The unsaturated substances formed by this procedure are as a general rule coloured, or form coloured compounds with atmospheric oxygen in a short time. This is perceived as a reduction in the product quality of the ester prepared. The unsaturated contents moreover often have the effect of a deterioration in the stability and durability of products to which these esters are added as an additive. The aggressiveness of the acid catalysts at elevated temperature additionally is a burden on the production plants. A usually low rate of reaction is considered to be a further disadvantage of the low temperature processes.

In the high temperature processes, organometallic complexes of the transition metals Ti, Zr, Al, Sn are conventionally employed as catalysts. Because of the high reaction temperature, however, still more coloured by-products are formed, so that expensive working up and/or purification processes become necessary. Furthermore, the removal of the catalyst from the end product is expensive.

EP 0 342 357 A2 describes a device and a process for carrying out esterifications. In this, esters are prepared from alcohols and fatty acids in a production plant at 200 to 250° C., the reaction mixture being led continuously over a particularly hot reaction zone with a short contact time and the preparation being carried out over reaction times of up to 20 hours.

With respect to industrial esterification reactions, there is need for improvement in various aspects in order to meet the requirements and demands of the market. The known processes have at least one, as a rule several of the disadvantages outlined below:
coloured nature or inadequate colourlessness of the products
undesirable by-products,
inadequate stability
low efficiency, high energy consumption, high production costs,
low yield,
impurities, in particular traces of heavy metals,
long reaction times.

There is therefore the need for improvement in the known processes and possibly the provision of new processes in order to be able to provide esters in an improved quality.

There is furthermore the demand for more efficient production processes or devices which have a lower consumption of energy and resources with high conversions, yields and selectivities and render after-treatment steps superfluous.

The present invention was based on the object of at least partly overcoming the disadvantages emerging from the prior art.

In particular, the present invention was based on the object of providing a process and a device with the aid of which by-products which differ from esters and play a part in the increase in the colour shading of the esters can be reduced, and in this way expensive and time-consuming purification steps can be reduced or even avoided.

A further object of the present invention was to provide additives for the preparation of thermoplastic compositions which, in addition to being environment-friendly, are suitable for modifying the properties of the thermoplastic composition in the desired manner and at the same time for obtaining thermoplastic compositions which meet high requirements, such as in the foodstuffs industry or in medicine.

A contribution towards achieving at least one of the abovementioned objects is made by the subject matter of the category-forming claims, the sub-claims dependent upon these representing further embodiments according to the invention.

The present invention provides a process for the preparation of an ester at least based on
a. at least one alcohol component,
b. at least one carboxylic acid component, and
c. optionally further additives,
as process components, comprising, in a reactor, the process steps:
i. provision of the process components,
ii. reaction of the process components to give an ester A,
iii. after-treatment of the ester A,
wherein the ester A is transferred into a working up container and combined there with
aa. at least one active component which is introduced into the ester A as a particulate solid, and
bb. optionally further auxiliary substances
cc. to give a mixture, before
dd. this mixture is divided into a solid and a liquid phase, the ester being obtained as the liquid phase.

In principle, any alcohol component with one or more hydroxyl groups which is known to the person skilled in the art and appears to be suitable for carrying out the process according to the invention is suitable as the alcohol component for carrying out the process according to the invention. The term “alcohol component” as used here includes the alcohol in its protonated form, the alcohol in its deprotonated form, in particular salts of the alcohol, and also mixtures of the alcohol in its protonated form and its deprotonated form or mixtures of the alcohol in its protonated form its deprotonated form and one or more salts of the alcohol.

According of a preferred embodiment of the present invention, less than 50 mol-%, preferably less than 40 mol-%, more preferably less than 30 mol-% and most preferably less than 20 mol-% of unreacted carboxylic acid is neutralized with a basic compound before ester A is combined with the at least one active component.

Alcohols with a number of hydroxyl groups in a range of from 1 to 9, particularly preferably 3 to 8 and most preferably 3 to 6 are preferably employed as the alcohol component with one or more hydroxyl groups. The number of carbon atoms in the alcohol with one or more hydroxyl groups is preferably in a range of from 3 to 30, particularly preferably 3 to 18, furthermore preferably 3 to 10 and most preferably 3, 4, 6 or 8.

A technical grade alcohol can also be employed as the alcohol component. “Technical grade” in connection with a chemical substance or chemical composition means that the chemical substance or the chemical composition contains small amounts of impurities. In particular, the chemical substance or the chemical composition can contain impurities in a range of from 5 to 20 wt. %, preferably from 5 to 15 wt. %, more preferably from 5 to 10 wt. %, based on the total amount of the chemical substance or chemical composition. Particularly preferably, the chemical substance or the chemical composition contains from 1.5 to 5 wt. % of impurities. Impurities are understood as meaning all contents which differ from the chemical substance or the chemical composition. For example, technical grade ethanol can contain from 5 to 8 wt. % of impurities. This example cannot be generalized for all alcohols, rather the content of impurities with respect to the classification as “technical grade” is substance- or composition-related, or also depends on the preparation process. This classification according to the substance and the preparation process is familiar to the person skilled in the art.

It is likewise conceivable that it is not an individual alcohol or an individual technical grade alcohol which is employed as the alcohol component, but a mixture of several alcohols in the context of the abovementioned chemical composition. For example, several forms of the alcohol in accordance with that stated above can be employed as a mixture. Preferably, several alcohols characterized by at least one of the following features, such as different number of carbon atoms, different number of hydroxyl groups or different structure, or alcohols which differ simultaneously in two or more of the above-mentioned features, such as can be obtained, for example, as technical grade products from large-scale industrial processes, are employed.

According to a preferred embodiment, monofunctional, difunctional, trifunctional, tetrafunctional or pentafunctional alcohols, or a mixture of two or more of these, are suitable as the alcohol component.

Alcohol components which are suitable in this connection are based, for example, on the following monofunctional alcohols: 1-propanol, 2-propanol, 1-butanol, 2-butanol, 2,2-dimethyl-1-propanol, 2-methyl-1-propanol, 2,2-dimethyl-1-propanol, 2-methyl-2-propanol, 2-methyl-1-butanol, 3-methyl-1-butanol, 2-methyl-2-butanol, 3-methyl-2-butanol, 1-pentanol, 2-pentanol, 3-pentanol, cyclopentanol, cyclopentenol, glycidol, tetrahydrofuryl alcohol, tetrahydro-2H-pyran-4-ol, 2-methyl-3-buten-2-ol, 3-methyl-2-buten-2-ol, 3-methyl-3-buten-2-ol, 1-cyclopropyl-ethanol, 1-penten-3-ol, 3-penten-2-ol, 4-penten-1-ol, 4-penten-2-ol, 3-pentyn-1-ol, 4-pentyn-1-ol, propargyl alcohol, allyl alcohol, hydroxyacetone, 2-methyl-3-butyn-2-ol, 1-hexanol, 2-hexanol, 3-hexanol, i-hexanol, 4-methyl-2-pentanol, 4-methyl-3-pentanol, 3-methyl-1-pentanol, 3-methyl-2-pentanol, cyclohexanol, 1-heptanol, 2-heptanol, 3-heptanol, 4-heptanol, i-heptanol, 5-methyl-2-hexanol, 5-methyl-3-hexanol, 5-methyl-4-hexanol, 4-methyl-1-hexanol, 4-methyl-2-hexanol, 4-methyl-3-hexanol, 3-methyl-1-hexanol, 3-methyl-2-hexanol, 1-octanol, 2-octanol, 3-octanol, 4-octanol, i-octanol, 2-ethylhexanol, 1-nonanol, 2-nonanol, 3-nonanol, 4-nonanol, 5-nonanol, i-nonanol, 1-decanol, 2-decanol, 3-decanol, 4-decanol, 5-decanol, 1-undecanol, 2-undecanol, i-undecanol, 1-dodecanol, 2-dodecanol, i-dodecanol, 1-tridecanol, 2-tridecanol, i-tridecanol, tetradecanol, hexadecanol, Guerbet alcohol, heptadecanol, 1-octadecanol, oleyl alcohol, eicosanol, behenyl alcohol, or two or more of these.

The following are suitable as the alcohol component based on difunctional alcohols: 1,2-ethanediol, 1,2-propanediol, 1,3-propanediol, dihydroxyacetone, 2-methyl-1,3-propanediol, 2-butyne-1,4-diol, 3-butene-1,2-diol, 2,3-butanediol, 1,4-butanediol, 1,3-butanediol, 1,2-butanediol, 2-butene-1,4-diol, 1,2-cyclopentanediol, 3-methyl-1,3-butanediol, 2,2-dimethyl-1,3-propanediol, 4-cyclopentene-1,3-diol, 1,2-cyclopentanediol, 2,2-dimethyl-1,3-propanediol, 1,2-pentanediol, 2,4-pentanediol, 1,5-pentanediol, 4-cyclopentene-1,3-diol, 2-methylene-1,3-propanediol, 2,3-dihydroxy-1,4-dioxane, 1,6-hexanediol, 2,5-hexanediol, 3,4-hexanediol, 1,2-hexanediol, 1,2-cyclohexanediol, 1,3-cyclohexanediol, 1,2-heptanediol, 1,7-heptanediol, 2,6-heptanediol, 3,4-heptanediol, 1,2-cycloheptanediol, 1,3-cycloheptanediol, 1,4-cycloheptanediol, 1,2-octanediol, 1,8-octanediol, 2,7-octanediol, 4,5-octanediol, 1,2-cyclooctanediol, 1,3-cyclooctanediol, 1,4-cyclooctanediol, 1,5-cyclooctanediol, 1,2-nonanediol, 1,9-nonanediol, 2-methyl-1,9-octanediol, 2,2-dimethyl-1,9-octanediol, or two or more of these.

The following are suitable as the alcohol component based on trifunctional alcohols: glycerol, 1,2,4-butanetriol, erythrose, threose, trimethylolethane, trimethylolpropane, 2-hydroxymethyl-1,3-propanediol or two or more of these.

The following are suitable as the alcohol component based on tetrafunctional alcohols: erythritol, threitol, pentaerythritol, arabinose, ribose, xylose, ribulose, xylulose, lyxose, ascorbic acid, glucosic acid γ-lactone, or two or more of these.

The following are suitable as the alcohol component based on pentafunctional and more highly functional alcohols: arabitol, adonitol, xylitol and dipentaerythritol.

According to a further preferred embodiment, the alcohol component is chosen from glycerol, glycerol dimer, glycerol trimer, glycerol tetramer, oligoglycerols, pentaerythritol, pentaerythritol dimer, pentaerythritol trimer, trimethylolpropane, bistrimethylolpropane, pentaerythritol, pentaerythritol dimer, n-butanol, i-butanol, n-propanol, i-propanol, 2,2-dimethylpropanol, 2-ethylhexanol, n-octanol, i-tridecanol, cetyl alcohol, stearyl alcohol, ethylene glycol, diethylene glycol, butyl glycol, dibutyl glycol, tributyl glycol, polyethylene glycol or two or more of these.

In this connection, reaction products of these alcohol components with ethylene oxide and/or propylene oxide are furthermore suitable, in each case independently between 2 and 30 units particularly preferably 2, 3, 4, 5, 6, 7, 8, 9 or 10 units of ethylene oxide and/or propylene oxide having been reacted on at least one, preferably two or more, particularly preferably all of the hydroxyl groups of the alcohol component. The use of di-, tri- or tetrabutyl glycol is moreover conceivable.

According to a further preferred embodiment, the alcohol component contains less than 10 wt. %, preferably less than 5 wt. % of nitrogen-containing compounds, based on the total weight of the alcohol component, nitrogen-containing compounds being both nitrogen-containing alcohol components and other nitrogen-containing organic compounds. The alcohol component furthermore preferably does not contain nitrogen atoms (N).

According to a further preferred embodiment, the alcohol component contains less than 10 wt. %, preferably less than 5 wt. % of aromatic ring compounds, based on the total weight of the alcohol component, aromatic ring compounds being both alcohols containing aromatic rings and other aromatic ring compounds. The alcohol component furthermore preferably does not contain aromatic ring compounds.

According to a further preferred embodiment, the alcohol component contains as non-metal atoms only non-metal atoms chosen from the group consisting of carbon (C), oxygen (O), nitrogen (N) or hydrogen (H) or several of these, preferably consisting of carbon (C), oxygen (O) and hydrogen (H).

In principle all carboxylic acids known to the person skilled in the art can be employed as the carboxylic acid component for the preparation of the ester. The term “carboxylic acid component” as used herein includes the carboxylic acid in its protonated form, the carboxylic acid in its deprotonated form, and also salts of the carboxylic acid, and also mixtures of at least two of the above, or of the carboxylic acid in its protonated form, its deprotonated form and at least one or more salts of the carboxylic acid.

The term “carboxylic acid component” furthermore in principle includes all compounds which contain at least one carboxylic acid group. This term also includes compounds which, in addition to the at least one carboxylic acid group, contain other functional groups, such as ether groups.

The term carboxylic acid component furthermore includes the acid halides, in particular chlorides of the carboxylic acid, and the acid anhydrides of the carboxylic acid. These carboxylic acid components preferably have an increased reactivity of the carboxylic acid group compared with the carboxylic acid, so that during a reaction with an alcohol the ester formation is promoted.

Preferably, carboxylic acid esters which are particularly preferably based on plant or animal oils or fats are employed as the carboxylic acid component, in particular the use of tallow, such as, for example, beef tallow, kidney tallow or bovine kidney fat, of lard, of fish oil, of neat's foot oil, of seed oil, such as, for example, argan oil, apricot kernel oil (marillen kernel oil), cottonseed oil, borage oil (borage seed oil), thistle oil, groundnut oil, hazelnut oil, hemp oil, rose-hip kernel oil, elder seed oil, jojoba oil, currant seed oil, coconut oil/coconut fat, kukui oil, kiwi seed oil, pumpkin seed oil, linseed soil, caineline oil, macadamia oil, almond oil, poppy oil, evening primrose oil, palm oil, palm kernel oil, peach kernel oil, rape oil, rice oil, castor oil, sea buckthorn kernel oil, mustard oil, nutmeg flower oil, sesame oil, shea oil/shea butter, soya oil, sunflower oil, walnut oil, grape seed oil, wheat germ oil or cedar oil, of fruit pulp fats, such as, for example, olive oil, palm oil, avocado oil or sea buckthorn oil, or also of germ oils, such as, for example, rape germ oil, wheat germ oil, maize germ oil, rice germ oil, rice husk oil, soya germ oil or sunflower germ oil, or mixture of two or more of these, being particularly preferred. The use of tallow, of rape oil and of coconut, canola, palm, soya or sunflower oil is most preferred.

If carboxylic acid esters are chosen, the process for the preparation of an ester is a transesterification.

A technical grade carboxylic acid can furthermore be employed as the carboxylic acid component.

It is likewise conceivable that it is not an individual carboxylic acid or an individual technical grade carboxylic acid which is employed as the carboxylic acid component, but a mixture of several carboxylic acids. For example, several forms of the carboxylic acid in accordance with that stated above can be employed as a mixture. Preferably, several carboxylic acids characterized by at least one of the following features, a different number of carbon atoms, a different number of carboxylic groups or a different structure, or carboxylic acids which differ simultaneously in several of the abovementioned features, such as can be obtained, for example, as technical grade products from large-scale industrial to processes, are employed. The substance-related amount of impurities in the technical grade is familiar to the person skilled in the art.

The use of mono-, di- or polycarboxylic acids is preferred according to the invention.

Possible carboxylic acid components are, in particular, saturated or unsaturated carboxylic acids, acid chlorides of the carboxylic acids and acid anhydrides of the carboxylic acids having a number of carbon atoms in a range of from 6 to 26, particularly preferably in a range of from 8 to 24, still more preferably in a range of from 10 to 22, moreover preferably in a range of from 12 to 20 and most preferably in a range of from 14 to 18. The carboxylic acid components furthermore preferably have from 8 to 12 C atoms.

Carboxylic acid components which are suitable in this connection are, for example, derived from the following monocarboxylic acids: acetic acid, propionic acid, butyric acid, valeric acid, caproic acid, oenanthic acid, caprylic acid (octanoic acid), i-octanoic acid, pelargonic acid (nonanoic acid), capric acid (decanoic acid), lauric acid (dodecanoic acid), myristic acid, palmitic acid, margaric acid, stearic acid, arachic acid, behenic acid or also unsaturated carboxylic acids, such as e.g. acrylic acid, methacrylic acid, 3-butenoic acid, 4-pentenoic acid, 5-hexenoic acid, 6-heptenoic acid, 7-octenoic acid, 8-nonenoic acid, 9-decenoic acid, undecylenic acid, palmitoleic acid, oleic acid, elaidic acid, vaccenic acid, icosenic acid, ricinoleic acid, 12-hydroxystearic acid, cetoleic acid, erucic acid, and polyunsaturated carboxylic acids, for example linoleic acid, linolenic acid, arachidonic acid, timnodonic acid, clupanodonic acid or cervonic acid.

Suitable carboxylic acid components based on dicarboxylic acids are, for example, malonic acid, maleic acid, fumaric acid, succinic acid, glutaric acid, adipic acid, pimelic acid, suberic acid, azelaic acid, sebacic acid, tartaric acid, malic acid, α-ketoglutaric acid, oxaloacetic acid, o-phthalic acid, m-phthalic acid or p-phthalic acid. Examples of a suitable tricarboxylic acid are trimellitic acid or citric acid. The use of a mixture of at least two of the abovementioned carboxylic acid components is furthermore conceivable.

According to a preferred embodiment, the carboxylic acid component is chosen from octanoic acid, i-octanoic acid, nonanoic acid, i-nonanoic acid, 9-decenoic acid, decanoic acid, i-decanoic acid, sebacic acid, palmitic acid, stearic acid, oleic acid, azelaic acid, HOOC—C₃₆H₇₂—COOH, phthalic anhydride.

According to a further preferred embodiment, the carboxylic acid component contains exactly one carboxyl group.

According to a further preferred embodiment, the carboxylic acid component contains less than 10 wt. %, preferably less than 5 wt. % of nitrogen-containing compounds, based on the total weight of the carboxylic acid component, nitrogen-containing compounds being both nitrogen-containing carboxylic acids and other nitrogen-containing organic compounds. The carboxylic acid component furthermore preferably does not contain nitrogen atoms (N).

According to a further preferred embodiment, the carboxylic acid component contains less than 10 wt. %, preferably less than 5 wt. % of aromatic ring compounds, based on the total weight of the carboxylic acid component, aromatic ring compounds being both carboxylic acids containing aromatic rings and other aromatic ring compounds. The carboxylic acid component furthermore preferably does not contain aromatic ring compounds.

According to a further preferred embodiment, the carboxylic acid component contains as non-metal atoms only non-metal atoms chosen from the group of carbon (C), oxygen (O), nitrogen (N) or hydrogen (H), carbon (C), oxygen (O) or hydrogen (H), or several of these.

According to a further preferred embodiment, the carboxylic acid component contains less than 10 wt %, preferably less than 5 wt % of compounds containing hydroxyl groups, based on the total weight of the carboxylic acid component, compounds containing hydroxyl groups being both hydroxycarboxylic acids, and other organic compounds containing hydroxyl groups. The carboxylic acid component furthermore preferably does not contain hydroxyl groups.

According to a further preferred embodiment, the carboxylic acid component comprises a mixture of adipic acid, or an adipic acid derivative, as a dicarboxylic acid, and at least one monocarboxylic acid.

“Pure” and “technical grade oleic acid” can be employed as oleic acid. A pure oleic acid is understood as meaning a composition which contains more than 98 wt. % of oleic acid. A “technical grade oleic acid” is understood as meaning a composition which contains oleic acid to the extent of 98 wt. % or less. Such a technical grade oleic acid contains e.g. oleic acid in a range of from 60 to 75 wt. %, linoleic acid in a range of from 5 to 20 wt. % and stearic acid in a range of from 0 to 5 wt. %, based on the total weight of the technical grade oleic acid, the sum of the percentages by weight being 100. A suitable technical grade oleic acid is marketed e.g. by Cognis Oleochemicals GmbH, Germany, under the name “Edenor TiO5”. Such a technical grade oleic acid which can preferably be employed can be obtained from animal fats, for example beef tallow. A technical grade oleic acid with a higher content of oleic acid can likewise be employed, e.g. with 80 to 95 wt. %, preferably 85 to 95 wt. % and furthermore preferably 90 to 95 wt. %, in each case based on the total composition. A technical grade oleic acid with 96 to 98 wt. % of oleic acid, based on the total composition, is very particularly preferred. Another technical grade oleic acid with approx. 80 to 90 wt % of oleic acid, 2 to 10 wt. % of linoleic acid, 2 to 6 wt. % of stearic acid and 2 to 6 wt. % of palmitic acid, based on the total weight of the other technical grade oleic acid, is furthermore preferred, the sum of the percentages by weight being 100. Such another technical grade oleic acid is marketed e.g. as “high oleic” sunflower oil or HO sunflower oil.

According to a further preferred embodiment, pentaerythritol dioleate can be prepared from oleic acid as the carboxylic acid component and pentaerythritol as the alcohol component. In addition to pentaerythritol and pure oleic acid, technical grades thereof can also be employed as educts in this process. If technical grades are employed, a product which contains at least 40, preferably at least 50, particularly preferably at least 60, and moreover preferably at least 70 wt. %, in each case based on this product, of pentaerythritol dioleate is usually obtained.

According to a further preferred embodiment, sebacic acid di(n-butyl) ester can be prepared from sebacic acid as the carboxylic acid component and n-butanol as the alcohol component.

According to a further preferred embodiment, stearic acid isobutyl ester can be prepared from stearic acid as the carboxylic acid component and 2-butanol as the alcohol component.

According to a further preferred embodiment, stearic acid butyl ester can be prepared from stearic acid as the carboxylic acid component and 1-butanol as the alcohol component.

According to a further preferred embodiment, a palm oil complex ester can be prepared from a mixture of adipic acid, palmitic acid and stearic acid as the carboxylic acid component and pentaerythritol as the alcohol component. Preferably, a mixture of 10 to 30 wt. % of adipic acid, 30 to 45 wt. % of palmitic acid and 40 to 50 wt. % of stearic acid, based on the carboxylic acid component, can be employed, the sum of the percentages by weight being 100. It emerges from this that a complex ester in the context of this invention is a mixture of two or more individual esters which, although generally present in the pure form, can occasionally contain small amounts of impurities which differ from esters.

According to a further preferred embodiment, trimethylol iso-nonanoate can be prepared from i-nonanoic acid as the carboxylic acid component and trimethylolpropane as the alcohol component. Instead of i-nonanoic acid, a C_{8/10}-carboxylic acid fraction which is obtainable in the preparation of fatty acids from plant or animal fats and in which the content of C₈- and C₁₀-carboxylic acid in each case independently is in a range of 40-50 wt. % can also be employed,

According to a further preferred embodiment, a complex ester I from 30 to 70 wt. % of i-nonanoic acid, 10 to 40 wt. % of a C_{8/10}-carboxylic acid fraction and 2 to 30 wt. % of dimer acid (mixture of isomeric dimers from C₁₈-fatty acids, Pripol™ 1022, Uniqema, Gouda, Holland) as the carboxylic acid component and 10-30 wt. % of pentaerythritol dimer as the alcohol component can be employed, the sum of the percentages by weight being 100.

According to a further preferred embodiment, a sebacic acid dioctyl ester can be prepared from sebacic acid as the carboxylic acid component and 2-ethylhexanol as the alcohol component.

The process according to the invention for the preparation of an ester from a carboxylic acid component and an alcohol component can be carried out in the presence of further additives, for example one or more catalysts, stabilizers, antioxidants, viscosity regulators and mixtures thereof.

The process according to the invention is preferably carried out in the presence of a catalyst. In principle, any compound which is known to the person skilled in the art and appears to be suitable for catalysis of the esterifications according to the invention is suitable here as the catalyst.

Preferably, the catalyst or a catalyst mixture is employed in a range of from 0.0001 to 5 wt. %, preferably from 0.0005 to 4 wt. %, further preferably from 0.001 to 3.5 wt. %, moreover preferably from 0.004 to 3.0 wt. %, in each case based on the total amount of process components a. and b. Particularly preferably, the amount of catalyst added is in a range of from 0.006 to 2.5 wt. %, from 0.008 to 2.2 wt. %, from 0.01 to 2.0 wt. %, from 0.03 to 1.8 wt. %, from 0.05 to 1.6 wt. % or from 0.08 to 1.3 wt. %, in each case based on the total amount of process components a. and b. A range of from 0.1 to 1.2 wt. %, from 0.2 to 1.1 wt. %, from 0.3 to 1.0 wt. % or from 0.4 to 0.9 wt. %, in each case based on the total amount of process components a. and b., is still more preferable. A weight content of catalyst or catalyst mixture of from 0.5 to 0.8 wt. % and from 0.6 to 0.7 wt. %, based on the total amount of process components a. and b., is equally preferable.

If the catalyst is a solid at room temperature, the catalyst is preferably present in the form of particles, for example in the ground form. A particle size in a range of from 10 μm to 2 mm, in particular 20 to 500 μm, is preferred here. In accordance with that stated above, preferably at least 40 wt. %, in particular at least 45 wt. %, particularly preferably at least 50 wt. %, and most preferably a range of from at least 40 wt. % to 60 wt. % of the particles, in each case based on the total weight of the catalyst, have a particle size in the ranges described above.

According to a further preferred embodiment, at least 70 wt. %, particularly preferably at least 80 wt. %, or at least 90 wt. %, up to 95 wt. %, or 98 wt. % of the catalyst, based on the total amount of catalyst, is present in the reactor. In accordance with that stated above, in a particularly preferred embodiment the catalyst is not configured as a fixed bed catalyst, or is not bound in a polymer matrix, or is not absorbed in a zeolite, or is not applied to a support surface.

At least one compound chosen from the group consisting of proton donor or electron donor, or both, can advantageously be employed as the catalyst.

Suitable catalysts from the group of proton donors are, for example, sulphuric acid or phosphoric acid, aliphatic or aromatic sulphonic acids, such as methanesulphonic acids or benzenesulphonic acids, such as o- or m-toluenesulphonic acid, particularly preferably p-toluenesulphonic acid or methanesulphonic acid. It is likewise conceivable to employ fluorinated aliphatic or aromatic sulphonic acids, particularly preferably trifluoromethanesulphonic acid.

Suitable catalysts from the group of electron donors are, preferably, metals, metal compounds or reducing acids. Suitable metals are, in particular, tin, titanium, zirconium, which are preferably employed as finely divided metal powders. Suitable metal compounds are the salts, oxides or soluble organic compounds of the metals described above, or a mixture of at least two of these. In contrast to the proton donors, the metal compounds are high temperature catalysts which as a rule achieve their full activity only at temperatures above 180° C. They are preferred according to the invention because a smaller amount of by-products, such as, for example, olefins, are formed compared with catalysis with proton donors. Catalysts which are particularly preferred according to the invention are a) one or more divalent tin compounds, or b) one or more tin compounds and elemental tin, which can react with the educts to give divalent tin compounds. For example, tin, tin(II) chloride, tin(II) sulphate, tin(II) alcoholates or tin(II) salts of organic acids, in particular of mono- and dicarboxylic acids, e.g. dibutyltin dilaurate, dibutyltin diacetate, or a mixture of at least two of these, can be employed as the catalyst. Particularly preferred tin catalysts are tin(II) oxalate and tin(II) octoate.

In principle all reducing acids which are known to the person skilled in the art and appear to be suitable are suitable as catalysts of the group of reducing acids. Hypophosphorous acid, sulphurous acid, oxalic acid, ascorbic acid, or two or more of these are particularly preferred.

According to a further preferred embodiment, a mixture which comprises at least two, in particular at least three catalysts from one or more of the above-mentioned groups is employed as the catalyst. Particularly preferably, two or more catalysts are chosen, each catalyst being chosen from in each case different abovementioned groups.

According to a further preferred embodiment, a catalyst mixture comprising at least two different catalysts is employed, the first catalyst being chosen from the group of proton donors and the at least one further catalyst being chosen from the group of electron donors, or a mixture of two or more of these. Such a catalyst mixture can have a high catalytic activity at temperatures which are lower compared with the high temperature catalysts, e.g. between 140 and 180° C., or between 120 and 185° C. At the same time, because of the lower process temperature, a smaller amount of by-products which appear coloured, in particular a smaller amount of substances which cause a yellowish or brownish colouring, is formed. A mixture comprising p-toluenesulphonic acid and a tin compound is particularly preferred as the catalyst mixture. According to a further preferred embodiment, the catalyst or the catalyst mixture does not contain tin oxide.

According to a further preferred embodiment, a mixture of 0.001 to 1 wt. % of an electron donor of the group of metal or metal compound, 0.001 to 1 wt. % of a proton donor and 0.001 to 1 wt. % of a second electron donor from the group of reducing acid, in each case based on the total amount of process components a. and b., can be employed as the catalyst. Particularly preferably, tin oxalate is chosen as the metal compound, p-toluenesulphonic acid is chosen as the proton donor and hypophosphorous acid is chosen as the reducing acid.

According to a further preferred embodiment, a catalyst which comprises one or more compounds chosen from the group consisting of sodium hydroxide, potassium hydroxide, lithium hydroxide, magnesium hydroxide, calcium hydroxide and strontium hydroxide is employed as an additive. Such a catalyst is particularly preferred if carboxylic acid esters are chosen in particular as the carboxylic acid component.

According to a further preferred embodiment of the process according to the invention, the at least one ester has between 1 and 6 ester groups.

In the context of carrying out the process according to the invention, process components a., b., d. and optionally c. are first employed in process step i. The sequence and the nature and manner of the addition of the individual components a., b., d. and optionally c. into the reactor in principle is not critical. Preferably, all the process components required for a reaction which are to be attributed to one of the groups chosen from alcohol component, carboxylic acid component and catalyst are in each case introduced into the reactor as process components within the particular group at least partly at the same time. In this context, the carboxylic acid components and alcohol components envisaged for the preparation of the ester according to the invention can be initially introduced and can then be reacted in the presence of a suitable catalyst or a suitable catalyst mixture. Furthermore, in a preferred embodiment the catalyst components are initially introduced together with one of the process components chosen from one of the groups of alcohol components or carboxylic acid components and the other components are then added. If the catalyst components are introduced into the reactor together with a process component, this can be effected by simultaneous introduction, and by introduction as a mixture, solution, suspension or dispersion.

Process components a., b., d. and the additives c. are provided in the reactor in liquid or in solid form. It may be preferable in the case of process components to be provided which are solid at the ambient temperature to be liquefied by heating. It is conceivable both that the liquefying is carried out in the course of providing the components, e.g. by means of a preheating stage, and that these process components are stored in liquid form at elevated temperature and are led from the holding place under thermostatic control and in an insulated line through a metering device. The addition of the process components in liquid form makes simple metering possible and promotes swift mixing of the process components introduced into the reactor.

Suitable metering devices are in principle all the devices which are known to the person skilled in the art and appear to be suitable. Electrically controllable shut-off valves or delivery pumps are particularly suitable.

The addition of the additives c. is in general carried out in a separate step to the components a., b. and d. already initially introduced. If these are solids, these are preferably introduced through a sluice at the upper side of the reactor, the contents of the reactor being stirred vigorously. A cellular wheel sluice can particularly preferably be employed as the sluice. It is often advantageous to mix the components, while stirring, in the context of providing them.

If at least one catalyst or a catalyst mixture is employed as an additive, a mixture of solids, a suspension or a liquid mixture is preferably employed. Preferably, the catalyst or the catalyst mixture is added only at the start of the reaction.

The reaction of the process components in process step ii. of the process according to the invention can be carried out by all processes which are known to the person skilled in the art and appear to be suitable. In this context, it may be advantageous to remove the water formed in the reaction from the reaction mixture, this removal of the water preferably being carried out by distillation, optionally by distillation with alcohol employed in excess, during the reaction.

Alcohol which has not reacted after the reaction has been carried out can also be removed from the reaction mixture, this removal of the alcohol preferably being carried out by means of distillation. When the reaction has ended, in particular after the unreacted alcohol has been separated off, the catalyst present in the reaction mixture can furthermore be separated off by washing with water, a filtration or by centrifugation, optionally after treatment with a base.

It is furthermore preferable to carry out the reaction at a temperature in a range of from 50 to 300° C., particularly preferably in a range of from 100 to 250° C. and very particularly preferably in a range of from 100 to 280° C., most preferably in a range of from 150 to 270° C. and furthermore preferably in a range of from 200 to 250° C. The preferred temperatures depend on the alcohol component chosen, the progress of the reaction, the catalyst type and the catalyst concentration. These can be easily determined by experiments for each individual case. Higher temperatures increase the rates of reaction and promote side reactions, for example splitting off of water from alcohols or the formation of coloured by-products or both.

It is furthermore preferable to carry out the reaction of the process components at a temperature in a range of from 50 to 160° C., particularly preferably in a range of from 80 to 150° C. and very particularly preferably in a range of from 100 to 140° C., most preferably in a range of from 120 to 140° C. Preferably, proton acids are then employed as the catalyst or catalyst mixture. Particularly preferably, no further catalysts are then added.

It is furthermore preferable to keep the process components uniformly mixed by stirring during the reaction.

According to a further preferred embodiment, a part of the process components is removed continuously from the reactor during the reaction, fed via a delivery line to an external flow-through heat exchanger and then fed back into the reactor. The external flow-through heat exchanger can be configured in any manner which is known to the person skilled in the art and appears to be suitable. Preferably, a plate heat exchanger, a tube bundle heat exchanger or a falling film evaporator or a combination of at least two of these, particularly preferably at least one falling film evaporator, can be employed as the flow-through heat exchanger. Furthermore, the outflow of the flow-through heat exchanger is connected to the reactor preferably via a return line of not more than 300 cm to 1 cm length, particularly preferably less than 200 cm to 10 cm length, most preferably less than 100 cm to 40 cm length. Particularly preferably, the outflow from the flow-through heat exchanger is connected directly, preferably via a flange, to the upper side of the reactor.

It is furthermore preferable in connection with the process according to the invention for the ester A obtained in the reaction ii. to be after-treated.

“After-treatment” is understood as meaning all conceivable steps and processes which are familiar to the person skilled in the art and which can be undertaken in order to purify the ester A obtained in process step ii. from by-products, impurities, catalysts and other additives or those processes with which the ester A is further processed to an end product.

These are understood as meaning, in particular, distillation, sorption, filtration, bleaching, centrifugation, washing, crystallization or drying processes, and continuing reactions, or a combination of at least two or more of these. Pressure filtration, bleaching and spray drying processes are preferred.

For the after-treatment, the ester A is transferred according to the invention into a working up container. This can be effected directly via a fluid-carrying connection, or via an intermediate stage, e.g. via a heat exchange zone. Such a heat exchange zone can be employed if the after-treatment is to be carried out at a temperature which differs from the temperature of the reaction. The temperature in the after-treatment is preferably in a range of from 10 to 200° C., particularly preferably from 50 to 100° C., further preferably from 60 to 90° C. and most preferably from 70 to 80° C. below the temperature in the reaction.

In the process according to the invention, the ester A is combined in the working up container with
aa. at least one active component, which is introduced into the ester A as a particulate solid, and
bb. optionally further auxiliary substances
cc. to give a mixture, before
dd. this mixture is divided into a solid and a liquid phase, an ester B being obtained as the liquid phase.

In principle, any active component which is known to the person skilled in the art and appears to be suitable for the after-treatment can be employed as the active component. A mixture of two or more active components can likewise also be employed. Active components in the context of the invention are understood as meaning in particular substances which can make a contribution towards improving the physical properties or the purity of the ester A prepared according to the invention or to both, without changing the identity of the ester A according to the invention by a chemical reaction. According to the invention, the active component is introduced into the ester A as a particulate solid.

According to a preferred embodiment of the process according to the invention, an amount of the active component in a range of from 0.01 to 20 parts to 100 parts of process components is introduced into the ester A. Further preferably, an amount of the active component in a range of from 0.05 to 10 parts, or from 0.1 to 5 parts, in particular from 0.2 to 3 parts, from 0.2 to 2 parts, or from 0.2 to 1 part, in each case to 100 parts of process components, is chosen. Still more preferably, an amount of the active component in a range of from 0.25 to 0.8 part, very particularly preferably from 0.3 to 0.7 part, most preferably from 0.4 to 0.6 part, that is to say, for example, 0.5 part, in each case to 100 parts of process components, is chosen. An amount of the active component in a range of from 0.5 to 1.0 part, in particular 0.7 to 0.8 part, in each case to 100 parts of process components, can occasionally also preferably be chosen.

In principle all particle sizes which are known to the person skilled in the art and appear to be suitable for the purpose of the present invention are possible as particle sizes of the active component introduced as a particulate solid. The solid is described as particulate in particular if at least some of its particles have a particle size of from 8 μm to 5 mm.

In accordance with that stated above, preferably at least 70 wt. %, in particular at least 80 wt. %, particularly preferably at least 90 wt. %, and most preferably a range of from at least 95 wt. % to 99.5 wt. % of the particles, in each case based on the total weight of the active components, have a particle size in a range of from 8 μm to 0.1 mm. The percentage by weight data described in the above sentence likewise in each case apply to the following particle size ranges: from 10 μm to 300 μm, or preferably from 10 to 100 μm, or from 10 μm to 50 μm, or 12 μm to 40 μm, or from 15 μm to 32 μm, in particular from 15 μm to 25 μm, in each case based on the total weight of the active component.

The active component present as a particulate solid can have particles of a single particle size, or particles of several particle sizes which form a particle size distribution. If a particle size distribution is present, a distribution which resembles a bell-shaped curve or corresponds to this is preferred.

It is furthermore also possible for agglomerates of particles to occur if two or more particles stick to one another. Such agglomerates are also included in the particle term according to the invention, regardless of the composition and formation thereof.

Furthermore, in one embodiment of the present invention the active component is chosen such that its fine dust content is as low as possible. The fine dust content is understood as meaning that weight content of particles which has a particle size of less than 8 μm. Preferably, the fine dust content is less than 30 wt. %, less than 20 wt. %, less than 10 wt. %, preferably less than 5 wt. %, or less than 0.5 wt. %, in each case based on the total weight of the active component. The fine dust content is often in a range of from 1 to 10 wt. %, based on the total weight of the active component.

According to a further preferred embodiment of the present invention, the active component has a BET surface area according to DIN 66131 in a range of from 0.50 to 1,500 m²/g. Active components with a BET surface area in a range of from 50 to 250 m²/g, from 70 to 190 m²/g, or from 90 to 140 m²/g are often preferred. A further preferred range lies in a range of from 850 to 1,100 m²/g, furthermore from 900 to 1,050 m²/g and particularly preferably from 950 to 1,000 m²/g.

Sorbents chosen from the group of inorganic silicon-oxygen compounds, active charcoal, kieselguhr, ion exchangers, or two or more of these are suitable as the active component. Inorganic silicon-oxygen compounds or active charcoal, or both, are preferably employed.

In the context of the present invention, the term “active charcoal” also includes active charcoal-carbon black, active charcoal-coke and graphite. Occasionally, however, non-active charcoal molecular sieves are employed as “active charcoal”. According to the invention, an active charcoal which is preferably chosen is one which consists to the extent of more than 80 wt. %, or more than 90 wt. %, or more than 95 to 99 wt. % of carbon, particularly preferably of elemental carbon. Such an active charcoal is particularly advantageous if it has a BET surface area in a range of from 800 to 1,100 m²/g, furthermore preferably from 850 to 1,050 m²/g, or from 900 to 1,050 m²/g, or from 900 to 1,000 m²/g.

In addition to a direct use as a sorbent, active charcoal can also be employed in combination with a further sorbent, or on a carrier material, or both. If the active charcoal is employed in combination with at least one further sorbent, the content of active charcoal can vary within a wide range, e.g. between 0.1 and 90 wt. %, based on the total weight of the sorbents. Preferably, the content of active charcoal is between 0.5 and 70 wt. %, in particular between 5 and 40 wt. %, based on the total weight of the sorbents. A uniform homogeneous distribution of the active charcoal is particularly advantageous.

Furthermore, sorbents coated with active charcoal can also be employed as the active component. In this case the active charcoal is at least partly combined with a carrier material. The carrier material can be either amorphous or crystalline or present in a mixed form of the two. Oxidic material can furthermore preferably be employed as the carrier material. Amorphous, preferably amorphous oxidic carrier materials, which can contain up to 50 wt. %, preferably 2 to 10 wt. %, based on the carrier material, of crystalline material, are particularly preferred. If crystalline contents, e.g. zeolite or aluminium phosphate, are present, the content thereof is advantageously in the range of from 0.5 to 50 wt. %, based on the total weight of the sorbents.

An inorganic silicon-oxygen compound, preferably a silicate, or two or more of these are suitable as the active component. The inorganic silicon-oxygen compound advantageously has a BET surface area of from 150 to 240 m²/g, particularly preferably from 180 to 220 m²/g, for example 195m²/g.

One or more compounds chosen from the group consisting of: silica, in particular in disperse or highly disperse form, kieselguhr, clay mineral, in particular montmorillonite or bentonite, or zeolites, or two or more of these, are preferably employed as the silicate.

Kieselguhr or bentonite, or both, are particularly preferably employed as the inorganic silicon-oxygen compound. Calcium bentonite is particularly preferably suitable as the bentonite, very particularly preferably acid-activated calcium bentonite.

A combination of two or more active components, in particular a combination of at least one inorganic silicon-oxygen compound and at least one active charcoal, or two or more of these, can furthermore be employed. If such a combination of at least one inorganic silicon-oxygen compound and at least one active charcoal is employed, a ratio of inorganic silicon-oxygen compound to active charcoal in a range of from 10:1 to 1:10, or from 5:1 to 1:5, or from 5:1 to 1:1, particularly preferably from 4:1 to 1.5:1 is advantageously employed. A combination of inorganic silicon-oxygen compound to charcoal compound in a range of 3:1 to 2:1 is very particularly preferred.

If kieselguhr is chosen as the active component, kieselguhr with a BET surface area of from 0.5 to 7 m²/g is preferred. Kieselguhr with a weight-average particle size of from 10 to 50 or from 20 to 40 μm is further preferred. The particle size can be determined with a Leeds & Northrup “X100 Microtrac particle size analyzer”.

Preferably, according to the invention, further auxiliary substances can be introduced into the working up container for after-treatment of the ester A. All substances which are known to the person skilled in the art and appear to be suitable can be chosen as auxiliary substances for this.

Suitable auxiliary substances are, for example, antistatics, antioxidants, anticaking agents, trickle agents, inhibitors, desiccants, rheology modifiers, or two or more of these.

A mixture of two or more of the abovementioned auxiliary substances which are assigned to the same or different abovementioned groups of auxiliary substances can furthermore be employed.

It is furthermore conceivable for the ester A, the active component or the auxiliary substance, or several of these, to have a content of a liquid. With respect to the liquid-containing substance, this liquid can have been introduced due to the preparation, from the environment or intentionally incorporated into the substance, e.g. in order to avoid release of dust during handling of a particulate solid. This is carried out, for example, if the active component is employed as a suspension.

According to a further preferred embodiment, the active component is introduced as a particulate solid with less than 5 wt. % of a liquid, based on the active component, into the ester A.

The substances transferred and introduced into the working up container are combined to form a mixture. This can be carried out in principle in any manner which is known to the person skilled in the art and appears to be suitable. For example, the substances can be mixed with a stirrer, by pumping the mixture in circulation or by introducing a gas into the lower region of the working up container. The mixture obtained in this way can be homogenized by further mixing, for example stirring.

Thereafter, the mixture is divided into a solid and a liquid phase, the ester being obtained as the liquid phase. Any method which appears to be suitable to the person skilled in the art can be employed for dividing the mixture. Filtration, pressure filtration, settling or centrifugation processes are preferred. Pressure filtration processes are particularly preferred. A pressure filtration process is understood as meaning such a process in which a mixture to be filtered is charged with pressure and is led into a filter device and divided on a filter surface, for example a narrow-mesh net, a filter paper, a woven fabric or a laid fabric. A filter press is preferred as the filter device. A pressure filtration process which is preferred according to the invention can be carried out under a pressure in a range of from 0.5 to 20 bar, preferably from 1 to 10 bar, furthermore preferably from 1.5 to 8 bar, likewise preferably from 2 to 7 bar, from 2.5 to 5 bar and most preferably in a range of from 3 to 4 bar. The pressure filtration process is often also carried out under a pressure in a range of from 1 to 3 bar.

It is further preferable according to the invention to carry out the division in the separating device at a temperature of from 60 to 100° C., preferably from 70 to 90° C. and most preferably from 85 to 90° C. The liquid phase thereby formed can be fed directly to a further process step, or at least partly fed back into the working up container in a circulation. Preferably, the after-treatment is carried out in a circulation for a certain duration. For example, the after-treatment can be carried out with a dwell time in a range of from 15 to 240 minutes, preferably from 30 to 120 minutes, further preferably from 40 to 90 minutes and particularly preferably from 50 to 60 minutes. A dwell time of from 40 to 90 minutes is preferred.

It may moreover be advantageous to carry out the after-treatment under increased pressure, at elevated temperature and over a certain dwell time, or under a combination of two or more of the abovementioned conditions.

In the context of the pressure filtration process described here, for the division the mixture can be led through a separating device with one or more filter chambers, the mixture being divided into a solid and a liquid phase in the one or the several filter chambers on one or more filter surfaces. Preferably, the separating device has at least two filter chambers. Particularly preferably, the separating device has in a range of from two to 50, further preferably in a range of from 5 to 30, or from 10 to 25, or from 15 to 20 filter chambers.

During the division of the mixtures, in the separating device a solid phase with a thickness in a range of from 1 to 20 mm, preferably from 2 to 18 mm, or from 4 to 15 mm, or from 5 to 12 mm, or from 6 to 10 mm or from 7 to 8 mm often forms in at least one of the filter chambers on at least one of the filter surfaces. Such solid phases can also be formed in several filter chambers or on several filter surfaces, or both. Preferably, solid phases are formed on all the filter surfaces in all the filter chambers of the separating device. It is furthermore conceivable for the mixture itself to be divided into at least two streams before the division into a solid and a liquid phase, for each stream to be divided into a liquid and a solid phase in its own separating device, and for the liquid phases obtained in this way then to be brought together again.

The solid phase preferably comprises the active component, preferably in an amount in a range of from 30 to 90 wt. %, further preferably from 50 to 80 wt. %, based on the total amount of solid phase.

When the after-treatment has ended, the ester B can be collected and made available in a holding unit.

In connection with the esters which can be prepared from a carboxylic acid component and an alcohol component with several hydroxyl groups in the process according to the invention, it is furthermore preferable for not all the hydroxyl groups of the alcohol component to be esterified, so that some of the hydroxyl groups remain non-esterified. In this connection, it is particularly preferable for from 5 to 80 mol %, particularly preferably from 10 to 70 mol %, still more preferably from at least 20 to 50 mol %, moreover preferably from 30 to 40 mol % and most preferably from 45 to 55 mol % of the hydroxyl groups of the alcohol component not to be esterified. This means that in the ester obtainable by reaction of the composition according to the invention, the content, described in mol %, of all the hydroxyl groups originally present in the alcohol component containing to several hydroxyl groups for the preparation of the ester from a carboxylic acid component and an alcohol component is not esterified, and thus is also present as hydroxyl groups in ester A, and optionally also in ester B.

The present invention also provides a device comprising as device units connected by fluid-conducting means
α) at least one educt reservoir,
β) a reactor with a mixing device,
γ) a working up unit,
wherein the working up unit comprises, connected by fluid-conducting means:
αα) a working up container,
ββ) a delivery pump and
γγ) a separating device, and
a filter press which has 2 or more filter chambers, at least two of these filter chambers being provided with a filter frame, and each filter frame being provided with a filter material, the filter material having a permeability to air of from 5 to 20 l·m⁻²·s⁻¹ and a weight per unit area of from 500 to 700 g/m², is employed as the separating device.

In principle, all reactor types known to the person skilled in the art which this person considers suitable for carrying out the process according to the invention can be employed. Preferably, a stirred tank on the side wall of which is arranged a heating jacket on the outside or inside is employed as the reactor. The heating jacket can be arranged on a part of the side wall or on the entire side wall. Preferably, the heating jacket is arranged on the entire side wall. Furthermore, the heating jacket particularly preferably can be controlled in sections. For example, the heating jacket is in 3, 4, 5 or more sections, each of which can be heated independently of each other. For transportation of heat, a heat transfer medium is led to the heating jacket through heating lines. All the usual heat transfer media known to the person skilled in the art are suitable as the heat transfer medium. The heat transfer medium can be either a heating means or a coolant. The heat transfer medium can also be under pressure. Preferably, heating steam, thermal oil or water, particularly preferably heating steam, is chosen as the heat transfer medium.

Furthermore, the reactor advantageously has a stirrer with a stirrer motor, transmission and stirrer shaft with stirrer blades, which is arranged on the upper side of the stirred tank, preferably centrally. The length of the stirrer shaft, the number of stirrer tiers arranged on the stirrer shaft, the diameter of these stirrer tiers and the geometry of the stirrer blades arranged in each stirrer tier are advantageously chosen such that during operation a uniform mixing of the process components, and where appropriate of the reaction products, is ensured, especially in the regions close to the base. The length of the stirrer shaft is preferably chosen such that the stirrer shaft extends from a motor lying outside the reactor, or from a transmission driven by a motor, almost to the base of the reactor. Preferably, the length of the stirrer shaft is chosen such that a distance of between about 5 to about 10%, with respect to the height of the reactor tank, remains between the end of the shaft and the reactor base. The stirrer shaft can be mounted on one side or, if the stirrer shaft is constructed to the reactor base, mounted at two points.

All stirrer types known to the person skilled in the art which this person considers suitable for carrying out the process according to the invention can be employed as stirrers. Preferably, stirrer types which have the effect at least in part of axial mixing during operation can be employed in particular. The stirrers can have one or more stirrer tiers, preferably one, 2, 3, 4, 5, 6 or 7 tiers. With respect to the geometry, cross, angled blade or disc stirrers with suitable stirrer blades are particularly preferred, and MIG or INTERMIG stirrers are most preferred. In the case of angled blade, disc and MIG stirrers, the stirrer blades in adjacent tiers can be displaced by 90° in the horizontal plane. The stirrers particularly preferably have an even number of tiers.

The stirrers are preferably produced from steel, preferably from V2A or V4A steel, particularly preferably from the following materials, the material numbers being found in EN10088: 1.4307, 1.4306, 1.4311, 1.4301, 1.4948, 1.4404, 1.4401, 1.4406, 1.4432, 1.4435, 1.4436, 1.4571, or 1.4429, particularly preferably 1.4301 or 1.4571.

The stirrer can moreover be at least partly coated with a surface coating composition. Preferably, the stirrer is equipped with a polymer coating. For example, a fluoropolymer coating which protects the material from which the stirrer is made from the fluid or mixture to be stirred is suitable as a polymer coating.

Preferably, a ratio of the diameter of the stirrer tier(s) to the diameter of the reactor of from 0.55 to 0.75, particularly preferably 0.60 to 0.70 or 0.62 to 0.68, very particularly preferably 0.64 to 0.66, e.g. 0.65, is chosen. By suitable choice of the parameters, the person skilled in the art ensures complete mixing and mingling in the reactor and avoids a deposit of solid constituents.

The stirrer blades can have the most diverse geometries, the geometry influencing the nature of the mixing. The “nature of the mixing” is understood as meaning the polar vector acting on the stirred mixture due to the movement of the stirrer. The polar vector has vertical and horizontal contents. Usually, both contents are not equal to zero. For example, a cross stirrer with stirrer blades arranged axially to the stirrer shaft and aligned vertical to the stirrer plane has the effect of rather horizontal mixing, whereas a cross stirrer with stirrer blades arranged at an angle, e.g. axially, to the stirrer shaft and at an angle of 30°, 45° or 60° with respect to the stirrer plane has the effect of a more vertical mixing. It is furthermore conceivable to provide a spiral stirrer.

Stirrers of which the stirrer blades have, with respect to the stirrer plane, a positive incline in the region close to the stirrer shafts, preferably the inner two thirds of the stirrer blade, and a negative incline in the region away from the stirrer shafts, preferably the outer third of the stirrer blade, are particularly preferred.

The incline of a stirrer blade is understood as meaning its alignment with respect to the stirrer plane, a positive incline meaning that the stirrer blade rises in the direction of rotation from its front edge from the bottom upwards to its rear edge, and has the effect of an ascending flow of material. A negative incline means that the stirrer blade drops in the direction of rotation from its front edge from the top downwards to its rear edge, i.e. has the effect of a descending flow of material. Such a stirrer has the effect of vertical mixing from the bottom upwards in the region of the middle of the reactor and a vertical mixture from the top downwards at the reactor wall.

The type of mixing described above can be assisted and adapted with further auxiliary devices. For example, baffles can be provided on the inside wall of the reactor. These are preferably attached to the inside wall of the reactor in the vertical direction, the plane in which the baffle lies being aligned through or at least in the direction of the vertical axis of the reactor.

According to another example, end stirrer organs which are moved a short distance above the reactor base can be attached on the lower stirrer tier. A short distance is to be understood as meaning so small that deposits of solid on the bottom can be carried along and/or swirled up by the stirrer. In this context, the end stirrer organs have the effect of a horizontal mixing to the extent of at least 50%, preferably 70%, based on the layer mixed by the end stirrer organs. The end stirrer organs preferably have a flat shape, the sides of the flat shape which are adjacent to the reactor base and the reactor wall being designed such that an essentially constant gap is provided between the reactor base and the reactor wall. If the reactor base is curved, for example, the end stirrer organs have a surface which is at least rounded at the side, and optionally an angled position of the end stirrer organs. Preferably, the end stirrer organs sweep over the reactor base at a distance of from 10 to 30 cm, preferably 15 to 25 cm or 30 cm.

In principle all materials which are known to the person skilled in the art and which this person considers suitable with respect to carrying out the process according to the invention, in particular with respect to strength, elasticity and corrosion resistance, can be employed as the material for production of the devices described above. In particular, the materials which are preferred in the choice of material for the stirrer are also preferred. Rustproof steel, preferably V2A or V4A steel, in particular the following materials, are preferably employed for production of the reactor, the material numbers being found in EN10088: 1.4307, 1.4306, 1.4311, 1.4301, 1.4948, 1.4404, 1.4401, 1.4406, 1.4432, 1.4435, 1.4436, 1.4571, or 1.4429, particularly preferably 1.4301 or 1.4571.

The device furthermore has a working up unit. Any device which is known to the person skilled in the art and appears to be suitable for improving a certain parameter of the crude product obtained in the reaction can be conceived as the working up unit. For example, a purification or separating device can be provided as the working up unit. Devices which have both a purifying and a separating action are usual in particular. Distillation units, filters, filter presses, sieves, separators, clarification devices or centrifuges, or a combination of two or more of these, are preferably suitable as working up units.

A line for removing a gaseous fluid stream which, for example, can remove by-products with a molecular weight of less than 100 g/mol is furthermore preferably provided on the reactor, this line being connected, if desired, to a pressure reducing unit for applying a reduced pressure. The fluid stream can furthermore be further treated, and for this led over at least one heat exchanger in order to cool the fluid stream. In this context, at least a part of the fluid stream can pass into a liquid phase, which is often collected and led back into the reactor or removed. This treatment of the fluid stream can be repeated twice or more often. If the fluid stream is led over at least two heat exchangers arranged in series, in the first heat exchanger a part of the fluid stream which differs from that in the at least second heat exchanger can pass into a liquid phase. It is thus possible, if desired, to lead a part of the fluid stream back into the reactor as a liquid phase and to discard another part of the fluid stream. Furthermore, the part of the fluid stream which is to be led back into the reactor as a liquid phase can optionally be divided into two immiscible phases in a separator with the aid of an adjustable removal device. Such a removal device is configured, for example, as an interfacial layer regulator. The first phase can then be passed back into the reactor via a return line. Alternatively, the entire fluid stream can be drained off and e.g. fed to another use, or discarded. The division of the fluid in the separator into two immiscible phases is carried out by appropriate alignment of the interfacial layer regulator. In principle any known embodiment which appears to be suitable to the person skilled in the art is suitable as the interfacial layer regulator.

it is furthermore conceivable to collect the fluid stream in a receiver before introduction into the separator, to lead the fluid stream over an additional heat exchanger and to further cool it in this way. At a lower temperature of the fluid stream, a better and faster demixing of at least two immiscible, liquid phases can be observed.

A heat transfer medium is led through each of the heat exchangers already mentioned. In order to effect cooling of the fluid stream, cooling fluids are preferably employed as heat transfer media. Preferably, the highest possible temperature difference is chosen between the fluid stream to be liquefied and the cooling fluid, in order to achieve marked cooling of the fluid stream. Furthermore, it may be entirely desirable to cool the fluid stream in a first step merely to a first temperature at which a part of the fluid stream is liquefied, before a further part of the fluid stream is liquefied in a downstream heat exchanger. It is conceivable that a first heat exchanger is operated with a cooling fluid which, for example, is at 20 or 25° C., or has a higher temperature, in order to separate off from the fluid stream at least a high-boiling content of the fluid stream which, e.g. has a boiling point in a range of from 80 to 120° C.

In this connection, a high-boiling content is understood as meaning one or more components of the fluid stream which have a boiling point in a range of from 50 to 150° C., preferably from 60 to 140° C., very particularly preferably from 70 to 130° C. In particular, a high-boiling content is understood as meaning those components which have a boiling point of from 80 to 160° C., in particular from 90 to 200° C. or more.

According to a further, preferred embodiment, the reactor can have on its under-side an outlet which is connected to a delivery pump by fluid-conducting means. to In principle, all pumps known to the person skilled in the art which this person considers suitable for carrying out the process according to the invention, taking into account the properties of the liquid to be delivered, which is optionally also in the form of a suspension, dispersion or emulsion, are suitable as the delivery pump. Preferably, a centrifugal, reciprocating, screw, impeller or hose pump can be employed. A centrifugal pump is very particularly preferred.

According to a further preferred embodiment, a delivery line from the delivery pump is connected to an external heat exchanger by fluid-conducting means, the external heat exchanger being connected to the reactor, preferably to the upper side thereof, by fluid-conducting means. The external heat exchanger is connected to the reactor preferably via a return line of not more than 300 cm to 1 cm length, particularly preferably less than 200 cm to 10 cm length, most preferably less than 100 cm to 40 cm length. Particularly preferably, the external heat exchanger is connected directly, preferably via a flange, to the upper side of the reactor. A plate or tube bundle heat exchanger or a falling film evaporator, or a combination of two or more of these, can be employed as the heat exchanger. A falling film evaporator is preferred.

By the use of an external heat exchanger, the introduction of energy into a delivery stream led via this can be established better both with respect to the duration of the introduction and with respect to the amount of energy, i.e. the heat supplied or removed. This form of introduction of energy renders possible a short duration of the introduction, and therefore little or no change at all to the substance treated in the heat exchanger, in the case of heat-sensitive substances, that is to say those which readily decompose or change. Furthermore, with the use of an external heat exchanger an advantageous ratio of heat transfer area in the heat transfer zone of the heat exchanger to reactor volume can be established.

The delivery stream is preferably led over the heat transfer surface as a film. In this case, the delivery stream has a low height above the heat transfer surface. This arrangement renders possible both a high and a uniform energy transfer rate, so that short energy introduction times are possible compared with other heat transfer arrangements or heat transfer devices. The exposure of the process components to heat in the delivery stream is therefore reduced. Furthermore, undesirable side reactions, e.g. oxidation or polymerization, can likewise be reduced or even avoided.

By suitable choice of the dimensions of the heat transfer surface, in particular of the zone in the flow direction swept over by the film, the volume throughput of the delivery stream and the amount of energy introduced into this can be adapted to the circulation throughput, and thus to the requirements of the process according to the invention. Preferably, a high ratio of heat transfer area in the heat exchanger to volume throughput of the delivery stream is chosen. Furthermore, a ratio of heat transfer area to volume throughput of the delivery stream in a range of from 15 to 1 h/m, particularly preferably from 5 to 1.1 h/m, further preferably from 2 to 1.3 h/m and most preferably from 1.7 to 1.4 h/m is preferred.

For example, the external heat exchanger can be configured as a falling film evaporator. In this case, on entry into the heat exchanger the delivery stream is divided and applied as a film to the inner surfaces of tubes connected to the falling film evaporator inlet by fluid-conducting means and preferably arranged side by side, the tube walls forming the heat transfer surfaces. The sum of the individual heat transfer surfaces of the individual tubes forms the heat transfer surface of the falling film evaporator. On passage of the divided delivery stream from the tube into the outflow of the falling film evaporator, the delivery stream is combined again.

The amount of energy per unit volume of the delivery stream which can be transferred in the heat exchanger is determined by the speed of the delivery stream, the distance flowed over in the direction of flow on the heat transfer surface and by the average thickness of the film when sweeping over the heat transfer surface. The thickness of the film means the height of the film over the heat exchanger surface. Preferably, the thickness of the film is in a range of from 2 to 20%, particularly preferably from 5 to 15%, and furthermore from 7 to 12%, in each case based on the internal diameter of the heat transfer surface constructed as tubes.

A further outlet can be positioned on the reactor under-side. The ester A can be removed from the reactor via this, e.g. by a second delivery pump, after the reaction has ended or been discontinued, and fed to a further processing stage, e.g. a filling unit, a heat exchanger, a processing and/or working up unit. Preferably, in the context of that said so far, the under-side of the reactor has an outlet via which both the delivery stream during the reaction and the ester A are led out of the reactor. In order both to be able to lead the process components over an external heat exchanger as a delivery stream during the reaction, and to be able to lead the ester A via the same outlet after the reaction in the reactor, a delivery pump on the outlet of which a distributing device is arranged is preferably provided on the outlet of the reactor. Several connections leading away are provided on this distributing device, at least a first connection being connected by fluid-conducting means to the external heat exchanger and a second connection to a feed to a further processing stage.

A working up unit is provided as a further processing stage. This has at least one working up container and optionally further devices. All embodiments which are known to the person skilled in the art and appear to be suitable are possible as the working up container. Preferably, the working up container has a tank with a stirrer and heating jacket, it being possible for the heating jacket to be arranged on the inside or outside.

All stirrer types which are known to the person skilled in the art and appear to be suitable for carrying out the process according to the invention can be employed as stirrers. Preferably, stirrer types which have the effect at least in part of axial mixing during operation can be employed. The stirrers can have one or more stirrer tiers, preferably one, 2, 3, 4, 5, 6 or 7 tiers. With respect to the geometry of the stirrer, propeller stirrers are preferred.

The working up unit furthermore has at least one feed, preferably on the upper side of the tank, which is connected to the reactor by fluid-conducting means. The tank furthermore has an outlet, preferably on its under-side. According to a preferred embodiment, a separating device is arranged in a fluid-carrying connection via a delivery pump. A liquid phase separated off in this separating device can be collected and led via a distributing device optionally via a return line into the tank of the working up unit in a circulation, or to a holding unit.

In principle all embodiments which are known to the person skilled and appear to be suitable for carrying out the process according to the invention can be employed as the separating device. For example, filters, centrifuges, separators or filter presses can be employed as the separating device. A filter press is preferably employed. A filter press often has, in a fluid-carrying arrangement, an inlet, at least two filter chambers and at least one recipient with an outlet. Preferably, the filter press has three or more filter chambers, preferably 4, 5, 6, 7, 8, 9, 10, 11, 12, 13, 14, 15, 16, 17, 18, 19, 20, 21, 22, 23, 24, 25, 26, 27, 28, 29 or 30 filter chambers, or a multiple thereof.

Preferably, at least two of the filter chambers are provided with at least one filter frame each. Advantageously, each filter frame is equipped with at least two layers, preferably at least three layer, or more than three layers. For example, a filter frame has a filter material as the first layer, a filter surface as the second layer, and optionally a filter cake as the third layer.

In principle all embodiments which are known and appear to be suitable to the person skilled in the art are possible as filter materials. Filter materials which have a permeability to air in a range of from 5 to 20 l·m⁻²·s⁻¹, preferably from 10 to 20 l·m⁻²·s⁻¹, or from 10 to 15 l·m⁻²·s⁻¹ are preferred according to the invention. The permeability to air can be determined in accordance with DIN 53887. Filter materials of which the weight per unit area is in a range of from 500 to 700 g/m² are furthermore preferred according to the invention. Filter materials which have both the preferred permeability to air, as described above, and the weight per unit area which is preferred according to the invention are furthermore preferred. Porous substances, paper, glass frits, porcelain frits, braids of metal wire, woven fabric or laid fabric of textile or plastics materials are preferably suitable as the filter material. Plastics materials which can be employed are e.g. knitted fabric of plastic based on polyamide, PET, PP, ETFE, PEEK, PVC, or a combination of two or more of these. A multifilament of PVC, polyamide 6, PP and PET is preferred as the filter material.

According to a further preferred embodiment, a filter surface, preferably a flexible filter surface, is arranged on at least one filter material, the filter surface being characterized by at least one of the following features:

FP1) a weight per unit area of 65-75 g/m², particularly preferably 68-72 g/m²;
FP2) a filtration speed of 20″-30″ according to DIN 53137,
FP3) a thickness of 24-30 mm,
FP4) a bursting pressure of 2.5-3.5 kp.

Furthermore, in embodiments according to the invention the filter surface has two or more of the above features. The following combinations of features represented with the aid of the combinations of figures thus result specifically as embodiments: FP1FP2, FP1FP3, FP1FP4, FP2FP3, FP2FP4, FP3FP4, FP1FP2FP3, FP1FP2FP4, FP1FP3FP4, FP2FP3FP4.

According to a further preferred embodiment, the filter surface is formed from, preferably bleached, cellulose. Further preferred embodiment of the filter surface comprise: polyesters, polyamides as fibre or thread material for filter cloth materials.

The working up unit furthermore comprises a filter cake, preferably in the separating device. This filter cake is present at least towards the end of the working up, and has a height of between 2 and 10 mm, preferably between 3 and 7 mm. The height here means the thickness of the filter cake perpendicular to the filter surface in the state loaded with liquid. If several filter cakes are present, e.g. if several filter chamber with filter surfaces are arranged side by side, the height of the several filter cakes is to be regarded as the arithmetic mean of the height of the individual filter cakes. In this case, the variation in the height when several individual filter cakes are considered is preferably less than 10%.

According to a further preferred embodiment, an active component is present in the working up container. Suitable active components and preferred embodiments correspond to those described above.

The reactor can furthermore have at least one educt reservoir. Any desired installations in which process components can be kept ready before the reaction are conceivable as the educt reservoir. A storage container, a tank, a boiler or a still is preferred. It is likewise possible to provide a storage container connected to a further production plant as the educt reservoir.

According to a further, preferred embodiment, the educt reservoir is connected to the reactor via a line which is led via a preheating stage. All devices known to the person skilled in the art which this person considers suitable for achieving this aim in carrying out the process according to the invention can be employed as the preheating stage. A plate or tube bundle heat exchanger, or a combination of two or more of these, is particularly suitable as the preheating stage. A tube bundle heat exchanger is preferred.

According to a further, preferred embodiment, the educt reservoir is temperature-controllable, for example by a heating jacket or cooling jacket for the educt reservoir. It may be desirable here for a substance which is solid at the ambient temperature to be kept ready above its melting point. If this substance is present in liquid form, simple, often also more accurate metering than e.g. in the case of metering of the solid is possible. Furthermore, by leading substances in closed lines, the risk of exposure and contamination of employees and the environment is avoided.

Preferably, the reactor is furthermore connected to a pressure reducing unit. This is preferably arranged in a fluid-carrying continuation of the heat exchanger or heat exchangers and is connected to the end of the line for removal and/or treatment of the fluid stream. In principle all units for generating a reduced pressure which are known to the person skilled in the art are suitable as the pressure reducing unit, as long as he would consider them taking into account the reactor design.

An embodiment, which also contains optional features and is in no way intended to represent a limitation of that said so far is explained further by way of example in the following with the aid of drawings.

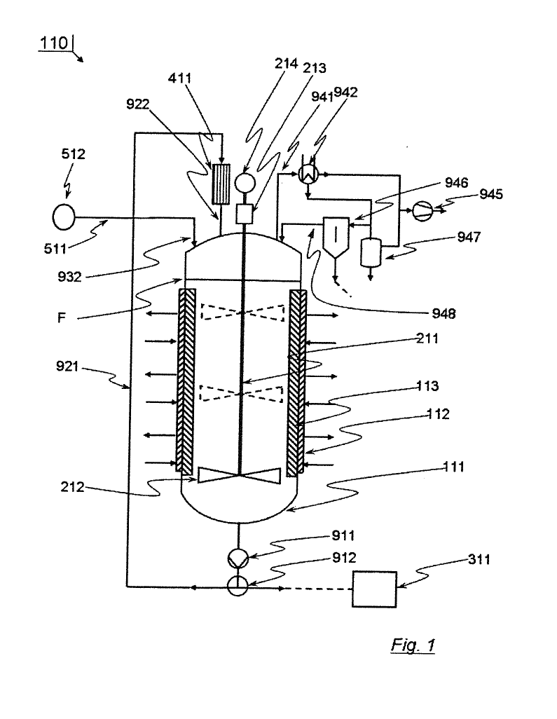

FIG. 1 shows a reaction region 110 with a reactor 111 with various installations which is suitable and preferred for carrying out the process according to the invention. The reactor 111 has on the reactor wall an external heating jacket 112. This is divided into three sections, which can be controlled separately. In the middle of the reactor, along its vertical axis, a stirrer 211 with stirrer tiers 212 is arranged. The stirrer 211 is driven via a transmission 213 with a motor 214. Baffles 113 can be arranged on the reactor wall. On the upper side of the reactor 111, an external heat exchanger 411 is arranged via a connection 922, which can be configured as a return line or as a flange. Preferably, the external heat exchanger 411 is configured as a falling film evaporator. On the under-side of the reactor 111 is an outlet with a shut-off valve, which is connected to a delivery pump 911. A distributing device 912, e.g. a multi-way valve, is attached to the outlet of the delivery pump. From the distributing device 921, a return line 921 leads to the external heat exchanger 411. A second line leads from the distributing device 912 to a working up unit 311. The filling level line F represents the position of the interface between the space underneath the interface taken up by the filling volume and the gas space above this. The ratio of filling volume to gas space and therefore the position of the filling level line F can vary between to reactor occupations, or at two points in time in the preparation process, or both.

On the upper side of the reactor 111, a feed line 511 is attached, which is connected to one or more educt reservoirs containing process components. Furthermore, a line 941 for removing a fluid stream leads from the upper side of the reactor 111 to the heat exchanger 942. This line is connected to a second heat exchanger 943. One or more cooling fluids flow through the heat exchangers 942 and 943 at the same or a different temperature. The exits of the heat exchangers 942 and 943 are connected to a receiver 947 and a separator 946. A condensate of the heat exchangers 942 and 943 can be fed either directly or via the receiver 947 and a further heat exchanger 944 to the separator 946. The separator 946 has an interfacial layer regulator, from which a return line 948 leads to the reactor 111. The separator 946 and the receiver 947 can likewise be emptied by an outlet in each case arranged on the under-side. A reduced pressure can be generated by a pressure reducing unit 945 via a line connected to the heat exchanger 944 and the receiver 947.

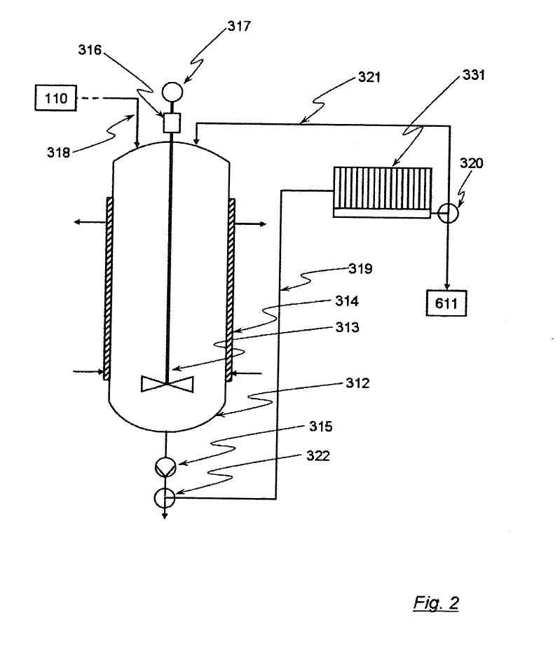

The working up unit 311, with a working up container 312 and a filter press 331, is shown by way of example in FIG. 2: From the reactor 111, a feed 318 which leads on the upper side of the working up container 312 into this is attached. The working up container 312 has a mixing device 313 driven by a motor 317 via a transmission 316. A heating jacket 314, externally here, is provided on the wall of the working up container. From the under-side of the working up container 312, a delivery line 319 leads via a delivery pump 315 to a filter press 331, the outlet of which is provided with a distributing device 320. From the distributing device 320, a return line 321 leads to the upper side of the working up container 312 and a further line leads to a holding unit 611.

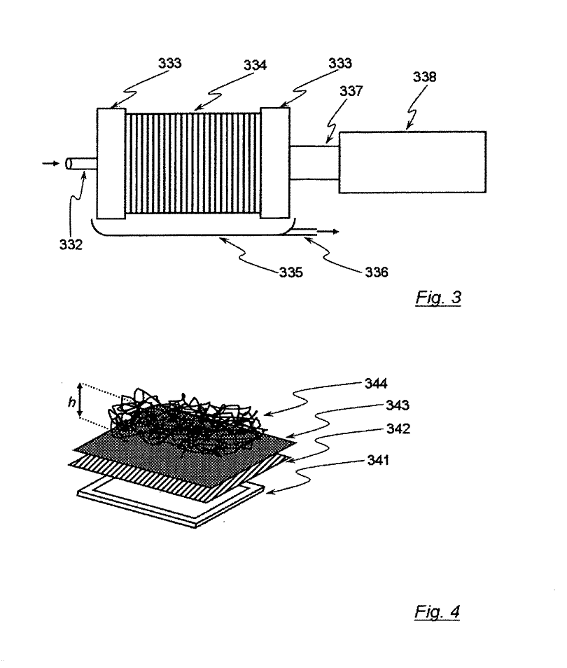

The filter press has the following components (FIG. 3): A feed line 332 is connected to a first front plate 333. Filter chambers 334 which are held together by a ram 337 driven by a motor with transmission 338 are arranged between the first front plate 333 and a further front plate 333. Under the filter press 221, at least under the filter chambers, a recipient 335 with an outlet 336 is arranged.

According to FIG. 4, each filter chamber 334 comprises a filter frame 341, on which are positioned a filter material 342 and a filter surface 343. Furthermore, a filter cake 344 of thickness (height) h can be positioned on the filter surface 343.

The present invention also provides a process for the preparation of a thermoplastic composition comprising
a1) a thermoplastic polymer,
b1) an additive, and
c1) optionally further additives,
   comprising the process steps:
i) provision of a thermoplastic polymer or of a precursor of a thermoplastic polymer or both;
ii) provision of an additive comprising an ester obtainable by the process according to the invention described above by reaction of at least one alcohol component and at least one carboxylic acid component, the device according to the invention described above preferably being employed;
iii) optionally provision of further additives,
iv) mixing of components i), ii) and optionally iii).

Those esters and further additives which have already been mentioned above as preferred esters and further additives in connection with the processes according to the invention for the preparation of an ester are preferred as esters and further additives.

In a preferred embodiment, the additive comprises a preferably at least partly hardened ester from a carboxylic acid component and an alcohol component with one or more hydroxyl groups.

“Hardened esters” in the present case are understood as meaning in particular vegetable esters in which the carboxylic acid components are derived from a carboxylic acid containing one or more double bonds. These double bonds can be at least partly or completely removed by hydrogenation. If not all of the double bonds of the carboxylic acid have been removed, a partly hardened ester is referred to, preferably at least 50 mol % and particularly preferably at least 70 mol % of the double bonds of the carboxylic acid having been hydrogenated, which can be determined, for example, by NMR spectroscopy or by determining the iodine number.

The term “thermoplastic polymer” is understood as meaning plastics which can be (thermo)formed (plastically) in a temperature range which is elevated with respect to room temperature. This operation is reversible and can be repeated by cooling and reheating into the molten state as often as desired, unless thermal decomposition of the material starts due to overheating.

Possible thermoplastic polymers which the composition according to the invention can contain are, in particular, thermoplastic polyurethanes, thermoplastic polyesters, thermoplastic polyamides, thermoplastic polyolefins, thermoplastic polyvinyl esters, thermoplastic polyethers, thermoplastic polystyrenes, thermoplastic polyimides, thermoplastic sulphur polymers, thermoplastic polyacetals, thermoplastic fluorinated plastics, thermoplastic styrene/olefin copolymers, thermoplastic polyacrylates, thermoplastic ethylene/vinyl acetate copolymers or mixtures of two or more of the abovementioned thermoplastic polymers.

It is preferable according to the invention for the thermoplastic polymer to be to based on thermoplastic polyesters to the extent of more than 90 wt. %, particularly preferably to the extent of more than 95 wt. %, moreover still more preferably to the extent of at least 99 wt. % and most preferably to the extent of 100 wt. %, in each case based on the total weight of the thermoplastic polymer. The term “polyester” as used herein includes in particular polymers which have been obtained by a polycondensation reaction between a polycarboxylic acid and a polyol (so-called “AA//BB polyesters”) or by a polycondensation reaction of a hydroxycarboxylic acid or by ring-opening polymerization of a cyclic ether (so-called “AB polyesters”). In one embodiment according to the invention, polycarbonates which are obtainable by reaction of phosgene with dials may be excluded from the term “polyester” used according to the invention.

In principle, all the thermoplastic polyesters and copolyesters currently known can be used as component a1) in the thermoplastic composition according to the invention. Examples of such polyesters include linear polyesters which have been prepared via a condensation reaction of at least one polycarboxylic acid, preferably a dicarboxylic acid (dibasic acid) or an ester-forming derivative thereof, and at least one polyol, preferably a dihydric alcohol (diol).

It is furthermore conceivable to prepare polyesters which have a degree of branching or crosslinking which is not equal to zero, that is to say are not linear.

In this connection, the degree of branching is the mean, over the sum of all the polyester molecules, of the ratio of the number of branching monomer units to the total number of all the monomer units per polyester molecule. The degree of branching is in a range of from 0.01 to 50 wt. %, preferably from 0.05 to 30 wt. %, further preferably from 0.01 to 20 wt. %, particularly preferably from 0.5 or 1 to 10 wt. % and most preferably between 3 and 7 wt. %, based on the sum of all the thermoplastic polyester molecules. If polyesters which are not exclusively linear but are branched to at least a small proportion, e.g. between 2 and 8 wt. %, are employed as the thermoplastic polyester, an adaptation of the physical properties of the thermoplastic composition, for example a reduction of the viscosity, can be established.

In this connection, the degree of crosslinking is the mean, over the sum of all the polyester molecules, of the ratio of the number of crosslinking monomer units to the total number of all the monomer units per polyester molecule. In this connection, the degree of crosslinking is in a range of from 0.001 to 3 wt. %, preferably 0.005 to 1 wt. %, particularly preferably 0.01 to 0.5 wt. % and most preferably 0.05 to 0.1 wt. %, based on the sum of all the thermoplastic polyester molecules. At these low degrees of crosslinking, the thermoplastic properties of the molecules are retained.

The preferably difunctional acid and the preferably difunctional diol can both be either aliphatic or aromatic, aromatic and partly aromatic polyesters being particularly preferred as thermoplastic moulding materials because of their high softening points and stability to hydrolysis. In the case of aromatic polyesters, between 80 and 100% of all the ester linkages are added on to the aromatic rings.

These thermoplastic moulding materials can be semicrystalline and even show liquid crystal properties or be amorphous. According to the invention, partly aromatic polyesters which have been obtained from at least one aromatic dicarboxylic acid or an ester-forming derivative thereof and at least one aliphatic diol are particularly preferred thermoplastic polyesters. Examples of suitable aromatic dicarboxylic acids include terephthalic acid, 1,4-naphthalenedicarboxylic acid or 4,4′-biphenyldicarboxylic acid. Examples of suitable aliphatic diols include alkylene diols, specifically those which contain 2 to 6 C atoms, preferably 2 to 4 C atoms, where ethylene glycol, propylene diols and butylene diols are to be mentioned in particular here. Ethylene glycol, 1,3-propylenediol or 1,4-butylenediol are preferably used as the polyol or diol component for the preparation of the thermoplastic polyesters contained as component a) in the composition according to the invention. Thermoplastic polyesters which are obtainable by reaction of a dicarboxylic acid with a diol and are particularly preferred according to the invention include, in particular, polyalkylene terephthalates, for example polyethylene terephthalate (PET), polypropylene terephthalate (PPT) or polybutylene terephthalate (PBT), polyalkylene naphthalates, for example polyethylene naphthalate (PEN) or polybutylene naphthalate (PBN), polyalkylene dibenzoates, for example polyethylene dibenzoate, and mixtures of at least two of these thermoplastic polyesters.

The partly aromatic polyesters described above can optionally contain a small amount of units which originate from other dicarboxylic acids, for example isophthalic acid, or other diols, such as cyclohexanedimethanol, which in general reduces the melting point of the polyester. A specific group of partly aromatic polyesters are so-called segmented or block copolyesters, which in addition to the abovementioned polyester segments (also called “hard segments”), contain so-called “soft segments”. These soft segments originate from a flexible polymer; that is to say one with amorphous contents with a low glass transition temperature (T_{g}) and low rigidity to the extent of 60 to 100 wt. %, preferably more than 70 and still more preferably more than 80 wt. % to 100 wt. %, based on the total weight of the polymer. This flexible polymer has reactive end groups, preferably two hydroxyl groups. Preferably, the glass transition temperature of these “soft segments” is below 0° C., particularly preferably below −20° C. and most preferably below −40° C. In principle, several different polymers can be used as the soft segment. Suitable examples of “soft segments” are aliphatic polyethers, aliphatic polyesters or aliphatic polycarbonates. The molecular weight of the soft segments can vary within wide limits, but is preferably between 400 and 6,000 g/mol.

In addition to the abovementioned linear polyesters which are obtainable via a polycondensation reaction of at least one polycarboxylic acid or an ester-forming derivative thereof and at least one polyol, the thermoplastic composition according to the invention can also contain thermoplastic polyesters which are obtainable by a polycondensation reaction of short-chain hydroxycarboxylic acids or by a ring-opening reaction of cyclic esters.

Examples of suitable short-chain hydroxycarboxylic acids which can be employed for the preparation of thermoplastic polymers include in particular L-lactic acid, D-lactic acid, DL-lactic acid, glycolic acid, 3-hydroxybutyric acid, 4-hydroxybutyric acid, 4-hydroxyvaleric acid, 5-hydroxyvaleric acid, 6-hydroxycaproic acid and mixtures of these hydroxycarboxylic acids. Examples of suitable cyclic esters include in particular glycolide (a dimer of glycolic acid) and ε-caprolactone (a cyclic ester of 6-hydroxycaproic acid).

The preparation of the thermoplastic polyesters described above is also described, inter glia, in “Encyclopedia of Polymer Science and Engineering”, volume 12, pages 1 to 75 and pages 217 to 256; John Wiley & Sons (1988) and also in “Ullmann's Encyclopedia of Industrial Chemistry”, volume A21, pages 227 to 251, VCH Publishers Inc. (1992). Thermoplastic polymers which are preferred according to the invention are polyethylene terephthalate (PET), polybutylene terephthalate (PBT) and polylactic acid (PLA), it being possible for a preferred embodiment of a thermoplastic composition of the present invention to contain each of this polymers in itself to the extent of more than 50 wt. %, preferably more than 75 wt. % and particularly preferably more than 90 wt. %, in each case based on the thermoplastic composition.

Components a1), b1) and optionally c1) are first provided in process steps i), ii) and optionally iii). The mixing of components i), ii) and optionally iii) is then carried out in process step iv) of the process according to the invention.

In this context, the mixing of components a1), b1) and optionally c1) can be carried out utilizing known techniques. Thus, the mixing can be, for example, a dry mixing operation, in which the various components are mixed at below the melt processing temperature of the thermoplastic polymer, or a melt mixing process, in which the components are optionally premixed, and mixed at the melt processing temperatures of the thermoplastic polymer. The melt mixing processes include, preferably, the melt kneading process, which can be realized, for example, by continuous melt kneading using a single-screw kneading machine, a twin-screw kneading machine of the toothed-same direction of rotation type, toothed-opposite direction of rotation type, nontoothed-same direction of rotation type, nontoothed-opposite direction of rotation type, or other types, or by batch melt kneading using a roller kneading machine, a Banbury kneading machine or similar. A combination of a dry mixing process and a melt mixing process is furthermore conceivable.

The sequence and the nature and manner of the addition of the individual components a1), b1) and optionally c1) into the mixing device is furthermore in principle not critical. Thus, for example, the thermoplastic polymer and optionally the additional substances can first be initially introduced into the mixing device and the additive only then added. It is also conceivable for the additive or a part of the additive first to be mixed with one or more other components of the thermoplastic composition according to the invention, for example with one or more additional substances, and then either for this mixture to be added to the thermoplastic polymer already in the mixing device, or for this mixture first to be initially introduced into the mixing device and the thermoplastic polymer only then to be added.

In further embodiments of the process according to the invention for the preparation of a thermoplastic composition, mixing is carried out in accordance with at least one of the following measures:
M1) at the glass transition temperature of the thermoplastic polymer or at a temperature above the glass transition temperature of the thermoplastic polymer;
M2) where the additive is more liquid than the thermoplastic polymer; or
M3) where at least a part of the additive is added to the precursor of the thermoplastic polymer.

Embodiments according to the invention furthermore include combining of two or more of the above measures. The following combinations of measures represented with the aid of the combinations of figures thus result specifically as embodiments: M1M2, M1M3, M2M3 and M1M2M3.

According to a preferred embodiment M1 of the process according to the invention, the mixing of components i), ii) and optionally iii) is carried out in process step iv) of the process according to the invention by a melt mixing process. In this connection it is preferable in particular for the mixing in process step iv) to be carried out at the glass transition temperature of the thermoplastic polymer or at a temperature above the glass transition temperature of the thermoplastic polymer. In this connection, it is particularly preferable for the mixing to be carried out at a temperature in a range of from 5° C. below the glass transition temperature (T_{g}) to 200° C. above the glass transition temperature of the thermoplastic polymer employed, particularly preferably at a temperature in a range of from 1° C. below the glass transition temperature (T_{g}) to 180° C. above the glass transition temperature of the thermoplastic polymer employed and most preferably at a temperature in a range of from 1° C. above the glass transition temperature (T_{g}) to 150° C. above the glass transition temperature of the thermoplastic polymer employed, the upper limit of the temperature range being essentially limited, however, by the decomposition temperature of the thermoplastic polymer employed. Embodiments according to the invention furthermore include mixing at temperatures in a range of from 10 to 180° C. and preferably 50 to 150° C. above the glass transition temperature of the thermoplastic polymer employed.

In embodiment M2 according to the invention, in which the additive is more liquid than the thermoplastic polymer, it is preferable for the additive to be employed at a temperature at which this is liquid and the thermoplastic polymer is not yet liquid. The temperature of the thermoplastic polymer here is preferably below the glass transition temperature of this polymer. It is thus preferable for the melting temperature of the additive and the glass transition temperature of the thermoplastic polymer to differ by at least 5° C.; preferably at least 10° C. and particularly preferably at least 30° C. In this embodiment and also generally, it is furthermore preferable for the thermoplastic polymer to be employed as granules. In general, all granule forms known to the person skilled in the art with a spherical or cylindrical spatial shape are also possible in the present case. The granule size, determined by means of sieve analysis, is in a range of from 0.01 to 5 cm, and preferably in a range of from 0.1 to 4 cm for at least 70 wt. % of the granule particles. By the procedure according to this embodiment, the surfaces of the granule particles can be at least partly coated with the additive according to the invention, so that at least partly coated thermoplastic polymer granules are obtained. This allows a distribution of the additive according to the invention in the thermoplastic composition which is as homogeneous as possible, especially if this is made up as a formulation for the extrusion taking place later.

In embodiment M3 according to the invention, in which the additive is added to the precursor of the thermoplastic polymer, additive in the liquid form and also in the solid form are possible. Possible precursors of the thermoplastic polymer are in principle all the precursors before the thermoplastic polymer is obtained which are known to the person skilled in the art. These include, in particular, precursors which have a lower molecular weight than the final thermoplastic polymer. It is preferable here for the molecular weight of the precursor to differ from that of the finished thermoplastic polymer by a factor of at least 1.1, preferably at least 1.5 and particularly preferably at least by a factor of 2. In addition to the monomers and oligomers, which preferably comprise 2 to 100 monomers, employed for the preparation of the thermoplastic polymer, a prepolymer which is polymerized completely, usually by heat treatment, to give the finished thermoplastic polymer is included, especially in the case of polycondensates. The prepolymer is preferably based on more than 100 monomers as recurring units, the number of the monomers as recurring units and therefore the final molecular weight of the finished thermoplastic polymer not being reached. It is therefore particularly preferable for the additive according to the invention in each case to be added to the monomers, oligomers or the prepolymer or at least two of these. By this means, in addition to a homogeneous distribution of the additive according to the invention, incorporation of the additive by chemical bonds with the thermoplastic polymer is also achieved, usually by the conditions prevailing during the polymerization or complete polymerization.

If the heated composition obtained in process step iv) in the case of a melt mixing process is not fed directly to the production of shaped articles, the process can also additionally include the further process step v):
v) cooling of the thermoplastic composition, preferably to a temperature in a range of from 20 to 30° C., particularly preferably to room temperature.

The thermoplastic composition which has been obtained in process step iv) can furthermore be fed to a granulation before, during or also after carrying out process step v), but optionally also after process step iv) and without carrying out process step v).

Furthermore, in addition to the thermoplastic polymer (component a1) and the additive (component b1), the thermoplastic composition according to the invention can optionally also contain further additives (component c1). The further additives include in particular impact modifiers, filler materials, reinforcing agents, flame retardant compounds, heat and UV stabilizers, antioxidants, other processing auxiliaries, nucleating agents, dyestuffs and antidripping agents. Examples of suitable impact modifiers, filler materials, reinforcing agents and flame retardant compounds are to be found, inter alia, in US 2005/0234171 A1.

It is furthermore preferable in connection with the process according to the invention for components a1) to c1) to be mixed with one another in relative amounts such that the thermoplastic composition obtained by mixing components a1) to c1) contains
a11) at least 40 to 99.99 wt. %, particularly preferably 50 to 99.8 wt. % and most preferably 60 to 99.6 wt. % of the thermoplastic polymer,
b11) 0.01 to 60 wt. %, particularly preferably 0.1 to 40 wt. % and most preferably 0.2 to 5 wt. % of the additive and
c11) 0 to 20 wt. %, particularly preferably 0.1 to 10 wt. % and most preferably 0.2 to 5 wt. % of the further additives
   in each case based on the total weight of the thermoplastic composition, wherein the sum of components a11) to e11) is 100 wt. %.

In another process embodiment according to the invention, it is preferable for components a12) to d12) to be mixed with one another in relative amounts such that the thermoplastic composition obtained by mixing components a12) to d12) contains
a12) 1 to 69.99 wt. %, particularly preferably 1.5 to 49.8 wt. % and most preferably 2 to 19.6 wt. % of the thermoplastic polymer,
b12) 0.01 to 20 wt. %, particularly preferably 0.1 to 10 wt. % and most preferably 0.2 to 5 wt. % of the additive,
c12) at least 10 wt. %, preferably at least 20 wt. % and particularly preferably at least 30 wt. % of a biodegradable filler component and
d12) 0 to 20 wt %, particularly preferably 0.1 to 10 wt. % and most preferably 0.2 to 5 wt. % of the further additives
   in each case based on the total weight of the thermoplastic composition, wherein the sum of components a12) to d12) is 100 wt. %.

Possible biodegradable filler components are in principle all those which are known to the person skilled in the art and appear to be suitable. These include, in particular, mono- and polysugars, such as starch and starch derivatives, cellulose and cellulose derivatives, hemp, jute, bast, rush, reed, in particular reed flour, and other substances obtained from plants or a combination of at least two of these. In the context of this embodiment, it is furthermore preferable for the thermoplastic polymer to be based on a monomer which can be generated from renewable raw materials, such as lactic acid, to the extent of at least 10 wt. %, preferably to the extent of at least 50 wt. % and particularly preferably to the extent of at least 75 wt. %, in each case based on the thermoplastic polymer. This thermoplastic composition is suitable in particular for biodegradable non-returnable and disposable articles, such as utensils or cutlery.

It is moreover preferable according to the invention for a saturated, α-olefinic oligomer of at least one C₆-C₁₈-α-olefin to be employed in the course of the process according to the invention for the preparation of a thermoplastic composition in at most an amount such that the thermoplastic composition obtained by mixing components a1) to c1) contains less than 0.001 wt. %, particularly preferably less than 0.0005 wt. % and most preferably less than 0.0001 wt. % of the saturated, α-olefinic oligomer.

A contribution towards achieving the abovementioned objects is furthermore made by the thermoplastic composition obtainable by the process described above. Here and generally, it is preferable for the thermoplastic composition to have a yellow value of less than 6.64, preferably less than 6, particularly preferably less than 5 and furthermore preferably less than 4 and moreover preferably less than 3. The yellow value is often less than 2, or than 1. In the ideal case it is 0, but often more than 0.1 or 0.2.

The present invention also provides a process for the production of a shaped article, comprising the process steps:
I) provision of a thermoplastic composition obtainable by the process described above for the preparation of a thermoplastic composition;
II) heating of the thermoplastic composition to the glass transition temperature of the thermoplastic polymer or to a temperature above the glass transition temperature of the thermoplastic polymer;
III) production of a shaped article from the heated thermoplastic composition prepared in process step II).

In step I) of the process according to the invention for the production of a shaped article, a thermoplastic composition according to the invention is first provided, this provision preferably being carried out by a process according to the process described above for the preparation of a thermoplastic composition.

In process step II), the thermoplastic composition is then heated to the glass transition temperature of the thermoplastic polymer or to a temperature above the glass transition temperature of the thermoplastic polymer. In this connection, it is in turn preferable for the heating of the thermoplastic composition to be carried out to a temperature in a range of from 5° C. below the glass transition temperature (T_{g}) to 100° C. above the glass transition temperature of the thermoplastic polymer employed, particularly preferably to a temperature in a range of from 1° C. below the glass transition temperature (T_{g}) to 50° C. above the glass transition temperature of the thermoplastic polymer employed and most preferably to a temperature in a range of from 1° C. above the glass transition temperature (T_{g}) to 20° C. above the glass transition temperature of the thermoplastic polymer employed, here also, however the upper limit of the temperature range being essentially limited by the decomposition temperature of the thermoplastic polymer employed.

In principle, process steps I) and II) can be carried out simultaneously or in succession. It is appropriate to carry out process steps I) and II) simultaneously, for example, if the thermoplastic composition is prepared by means of a melt mixing process. Where appropriate, it may be advantageous here to convert the composition prepared by the melt mixing process directly into a shaped article. It is appropriate to carry out process steps I) and II) successively, for example, if the thermoplastic composition is prepared by means of a dry mixing process or if the thermoplastic composition is indeed prepared by means of a melt mixing process, but is not subjected to the formation of a shaped article directly after the preparation, but rather is first cooled according to process step v).

In process step III) of the process according to the invention for the production of a shaped article, a shaped article is produced from the heated thermoplastic composition prepared in process step II). Possible processes for the production of a shaped article are, in particular, injection moulding, extrusion moulding, compression moulding, layer moulding, laminating moulding, blow moulding, vacuum moulding and transfer moulding, injection moulding being particularly preferred.

Furthermore, in a preferred embodiment of the process according to the invention for the production of a thermoplastic shaped article, in at least one further process step IV) at least a part region of the shaped article obtained in process step III) serves as a shaped article blank and is reduced in its mass cross-section compared with process step III). The mass cross-section is the cross-section of a region of the shaped article made solidly from the thermoplastic moulding composition according to the invention. For example, in containers or vessels, the mass cross-section is the thickness of a wall of these containers or vessels. In the case of shaped articles which are rather thread- or strand-like in construction, the mass cross-section is the thickness of these threads or strands. In the case of rather planar structures, such as sheets, layers, webs, films or foils, the mass cross-section is the thickness of these planar structures. For the reduction in the mass cross-section, in principle all the methods known to the person skilled in the art and suitable for this are possible. These include, for example, stretching in one or two directions, drawing in one or two directions, centrifugation or blowing, each of which is preferably carried out at elevated temperatures at which the thermoplastic composition according to the invention is so soft or even liquid that stretching, drawing, centrifugation or blowing can be carried out. The part region in which the reduction in cross-section is effected preferably makes up at least 50% and particularly preferably at least 80% of the shaped article obtained in step III). Stretching or drawing are generally carried out if a fibre is to be obtained from the shaped article obtained in step III). For the production of films, on the one hand drawing or stretching in one or more dimensions can be carried out. Thus, the web running out of an extruder can be drawn on to a roll at a higher speed compared with the exit speed from the extruder. On the other hand, if a container or vessel is to be obtained, apart from stretching, drawing and centrifugation, blowing is chiefly employed in step IV). In this, the reduction in mass cross-section is effected by applying a gas pressure. The gas pressure is generally chosen such that the thermoplastic composition, which is usually heated at least to the glass transition temperature, of the shaped article obtained in step III) can be extended. The extending is as a rule limited by using a mould having the final shape of the shaped article. In addition to containers, such as freezer boxes, dishes and packaging for foodstuffs, such as fruit, vegetables or meat, as well as medicaments as tablets, capsules, suppositories or powders, vessels for liquids can also be produced in this way. As well as for liquids of the cosmetics or pharmaceuticals industry, these vessels for liquids can also be used in the foodstuffs industry, preferably in the drinks industry, as reusable vessels, such as PET or PLA bottles. It is furthermore possible for two or more of process steps I) to IV) to be supplemented by further process steps and/or to at least overlap in time. This applies in particular to process steps III) and N).

In addition to bottles, other shaped articles can furthermore also be produced according to the invention. These include disposable and reusable vessels, such as plates, dishes, pots or beakers, and cutlery, such as knives, forks or spoons. The biodegradable thermoplastic compositions according to the invention are particularly suitable for these uses.

The present invention also provides a process for the production of a packed product, comprising the process steps:
a) provision of a product and a shaped article, in particular a film, the shaped article being obtainable by the process described above;
b) at least partial surrounding of the product with the shaped article.

The product provided in process step a) is preferably a pharmaceutical, a body care composition, an agricultural auxiliary substance, an adhesive, a building material, a dyestuff or a foodstuff.

The at least partial surrounding of the product can be carried out, for example, by the process described in DE-A-103 56 769.

The present invention also provides a process for the production of an at least partly coated object, comprising the process steps:
a4) provision of a coating composition comprising at least 10 wt. %, based on the coating composition, of an ester or a thermoplastic composition obtainable by the particular process described above, and of a solid substrate;
b4) mixing of the coating composition and the substrate, wherein the coating composition is at least partly liquid.

The production of the at least partly coated object with the coating composition can be carried out, for example, by a procedure in which the coating composition and the object to be coated are mixed with one another in suitable mixing devices, the Patterson-Kelly mixer, DRAIS turbulence mixer, Lödige mixer, Ruberg mixer, screw mixers, plate mixers and fluidized bed mixers being possible in particular as mixing devices. Should the coating composition not be liquid under the mixing conditions, this component is to be heated to a temperature above the melting temperature of the coating composition before or during the mixing with the object to be coated. In addition to the use of the mixing devices described above, the production of the at least partly coated object can also be carried out by a procedure in which, for example, the object to be coated is initially introduced into a fluidized bed mixer and the coating composition is sprayed in liquid form on to the object to be coated.

The present invention also provides a further processing product comprising an ester which is obtainable by reaction of a carboxylic acid component with an alcohol component which is capable of reacting with the carboxylic acid component to form an ester, and at least one functional component chosen from the group consisting of thermoplastic polymer, enzyme, curing agent of an adhesive, paraffin, oil, colouring agent, hair or skin care substance, polymer dispersion, lime mud, lubricant or emulsifier, or a combination of two or more of these.

The present invention also provides the use of an ester which is obtainable by reaction of a carboxylic acid component with an alcohol component which is capable of reacting with the carboxylic acid component to form an ester, as an additive in a composition which is chosen from the group consisting of thermoplastic composition, detergent, adhesive, defoamer, lubricant formulation, lacquer, paint, cosmetic formulation, soil compacting agent, drilling mud, hydraulic oil or dispersion.

According to a further preferred embodiment, the ester described above is used as an additive in a composition comprising as a functional component
α) a thermoplastic polymer, the composition being a thermoplastic composition;
β) an enzyme, the composition being a detergent;
γ) a curing agent of an adhesive, the composition being an adhesive;
δ) a paraffin, the composition being a defoamer;
ε) an oil, the composition being a lubricant formulation;
ζ) a colouring agent, the composition being a lacquer or a paint; or
η) a hair or skin care substance, the composition being a cosmetic formulation,
θ) a polymer dispersion, the composition being a soil compacting agent,
τ) a lime mud, the composition being a drilling mud,
κ) a lubricant, the composition being a hydraulic oil,
λ) an emulsifier, the composition being a thermoplastic composition or a dispersion,
   wherein the ester has preferably been obtained by the process according to the invention described above for the preparation of an ester comprising process steps i., ii. and optionally iii.

Preferably, the additive is employed in an amount in a range of from 0.001 to 40 wt. %, particularly preferably in a range of from 0.01 to 20, very particularly preferably from 0.1 to 10 wt. % and particularly preferably in a range of from 0.5, 1 or 2 to 5, 6, 7 or 8 wt. %, based on the composition.

The invention is now explained in more detail with the aid of non-limiting examples.

### Measurement Methods

Unless expressly stated otherwise, all the measurements are carried out in accordance with the relevant ISO standards. Unless specified otherwise there, a temperature of 23° C., an atmospheric pressure of 1 bar and a relative atmospheric humidity of 50% was chosen.

### Composition of a Mixture of Several Carboxylic Acid Components

Mixtures of several carboxylic acid components such as are present, for example, in technical grade oleic acid can be determined by means of gas chromatography (GC) or high pressure liquid chromatography (HPLC). The weight contents are stated in wt. %, based on the total weight of the sample supplied.

### Determination of the Yellow Value

As a measure of the yellow shading/yellowing, the yellow value is determined in accordance with DIN 5033 as the b* value of a sample of the composition to be analysed according to the L*,a*,b* colour system.

### Thermal Analysis

The thermal parameters were determined by means of DSC (dynamic heat flow differential calorimetry) on a Mettler Toledo DSC 821. The amount weighed out was 20-25 mg in an open crucible. After closing the crucible, the samples prepared in this way were measured over a temperature range of from −100 to +120° C. at a heating rate of 20 K/min against an empty, likewise closed crucible. The sample was kept at +120° C. for 60 seconds and then cooled with the highest possible cooling rate of the apparatus, but at least 15 K/min, to the abovementioned minimum temperature, and kept at this for 2 min before the heating and cooling operation was repeated.

The glass transition temperature and the melting temperature are determined in accordance with DIN 53765.

The degree of crystallization can be determined with the aid of the formula: K=ΔHₘ/ΔHₘ^{o}×100%, where K represents the degree of crystallization, ΔHₘ represents the enthalpy of fusion of the sample and ΔHₘ^{o} represents the enthalpy of fusions of the material at a crystallinity of 100%.

The ΔHₘ^{o} values can be found by the person skilled in the art in the tabular works known to him, e.g. J. Brandrup et al., “Polymer Handbook”, 4th ed., John Wiley & Sons.

### Further Methods

The following characteristic values are determined in accordance with published standards:

| Characteristic value | Standard | Comments |
|---|---|---|
| BET surface area | DIN 66131 | with nitrogen |
| Hydroxyl number (OHN) | DIN 53240 | |
| Acid number (AN) | DIN EN ISO 2114 | |
| Saponification number (SN) | DIN 53401 | |
| Pour point | DIN ISO 3016 | |
| Cloud point | DIN ISO 3015 | |
| Glass transition temperature | DIN 53765 | see above |
| (T_{g}), melting point (Tₘ) | | |
| Density | DIN 51757 | at 20° C. |
| Viscosity | DIN 53015 | at 20° C. |
| Gardner colour number | DIN EN ISO 4630-1 | |
| Thickness of the multifilament | DIN 53855 Part 1 | |
| [μm] | | |
| Particle size by means of laser | ISO13320-1 | with Coulter 230 LS |
| diffractometry | | |
| Water content | DIN 51777 | Karl-Fischer method |

### EXAMPLES

Unless noted otherwise, the raw materials, obtainable under the trade names given, are obtainable from Cognis Oleochemicals Deutschland GmbH, Düsseldorf, or from Sigma-Aldrich Chemie GmbH, Steinheim.

### Example 1

### Preparation of Pentaerythritol Dioleate

621.4 g of technical grade oleic acid (2.2 mol) and 136 g (1 mol) of pentaerythritol were initially introduced into a glass flask and 0.2 g of tin oxalate was added. The mixture was heated under reflux from 130 to 180° C. in the course of 3 hours and at 180° C. for a further 4 hours. After the mixture had been cooled, this was washed 8 times with 10 g of water and the pentaerythritol dioleate obtained in this way was dried in vacuo (p approx. 16 mbar) at 95-100° C. for 3 hours. Finally, 30 g of kieselguhr (“Hyflo Super Cel”, obtainable from Carl Roth) was stirred into the pentaerythritol dioleate and the suspension obtained in this way was filtered into a suction flask. The filtrate was dried again at 100° C. in vacuo (p approx. 16 mbar) over a period of one hour.

A yellowish liquid was obtained; the yield was 87%, based on the pentaerythritol weighed out. The acid number was 1.2.

### Example 2

### Preparation of Oleic Acid Glyceride with a Tin Catalyst

In accordance with Example 1, 621.4 g of technical grade oleic acid (2.2 mol), and 92.1 g (1 mol) of glycerol and 0.1 g of tin oxalate were initially introduced into the reaction vessel and reacted as in Example 1. The cooled reaction mixture was washed three times.

The yield was 79 wt. %, based on the glycerol weighed out. The oleic acid glyceride obtained in this way comprised glycerol dioleate to the extent of 63 wt. % and 16% of glycerol monooleate.

### Example 3

### Preparation of Oleic Acid n-octyl Ester

313.7 g (2.41 mol) of n-octanol and 421 g (1.5 mol) of technical grade oleic acid (EDENOR TiO5) were initially introduced into a flask with a distillation bridge and 0.38 g of tin oxalate was added. The reaction mixture was heated from 150° C. to 180° C. in the course of 3 hours. Thereafter, a vacuum was slowly applied and, after a further 4 hours at 180° C. (acid number of the reaction mixture=1.0), the excess n-octanol was distilled off in vacuo (p approx. 16 mbar). The mixture was cooled to 90° C., 25 g of kieselguhr (“Tonsil Optimum FF”, manufacturer Südchemie, Munich) were stirred in and the suspension obtained in this way was filtered into a suction flask.

The yield was 91%, based on the technical grade oleic acid weighed out.

### Example 4

### Preparation of Sebacic Acid Di(n-Butyl Ester)

176 g (2.41 mol) of n-butanol and 202 g (1.0 mol) of technical grade sebacic acid were initially introduced into a flask with a distillation bridge and 0.38 g of tin(II) oxalate was added. The reaction mixture was heated from 150° C. to 165° C. in the course of 3 hours. Thereafter, a vacuum was slowly applied and, after a further 4 hours at 165° C. (acid number of the reaction mixture=0.5), the excess n-butanol was distilled off in vacuo (p approx. 13 mbar). The mixture was cooled to 90° C., 25 g of kieselguhr (“Tonsil Optimum FF”, manufacturer Südchemie, Munich) were stirred in and the suspension obtained in this way was filtered into a suction flask.

The yield was 87%, based on the technical grade sebacic acid weighed out.

### Example 5

### Preparation of Sebacic Acid Di(n-Butyl Ester)

163 g (2.2 mol) of n-butanol and 202 g (1.0 mol) of technical grade sebacic acid were initially introduced into a flask with a distillation bridge and a mixture of 0.22 g of hypophosphorous acid, 0.22 g of tin(II) oxalate and 0.38 g of p-toluenesulphonic acid was added. The reaction mixture was heated at 150° C. for 20 hours, during the last 5 hours a vacuum being applied, starting at 500 mbar to 2 mbar. The mixture was filtered at 90° C. as in Example 4.

The yield was 93%, based on the technical grade sebacic acid weighed out. The acid number was 0.2.

### Example 6

### Preparation of Sebacic Acid Di(1-Butyl Ester)

The procedure was as in Example 4, i-butanol being initially introduced as the alcohol.

The yield was 91%, based on the technical grade sebacic acid weighed out.

### Example 7

### Preparation of Sebacic Acid Dioctyl Ester

The procedure was as in Example 4, n-octanol being initially introduced as the alcohol.

The yield was 83%, based on the technical grade sebacic acid weighed out.

### Example 8

### Preparation of Complex Ester I

650 g of i-isononanoic acid, 300 g of a C₈/C₁₀-carboxylic acid fraction, 100 g of dimer acid (Pripol™ 1022, Uniqema, Gouda, Holland) and 180 g of pentaerythritol dimer were heated, after addition of 5 mg of tin oxalate, at 190° C. for 30 hours in a 5 l reactor with an attached water separator.

After cooling to 50° C., the complex ester I obtained in this way was washed three times with 50 ml of water and filtered over 5 g of kieselguhr.

The purified complex ester I obtained in this way has an acid number of 0.7 and a Gardner colour number of 4.5.

### Example 9

### Preparation of a Thermoplastic Composition

6 kg of polyethylene terephthalate (PET SP04 from Catalana de Polimers, Barcelona, Spain) were introduced into a 15 kg Henschel mixer. The mixing wall temperature was 40° C. 0.5 wt. % of the ester prepared in Example 3 was furthermore added as a mould release agent. The material was then granulated on a granulator (ZSK 24Mcc) with a stuffing screw.

### Example 10

### Production of a Shaped Article

For production of shaped articles from the thermoplastic composition prepared in Example 9, a fully hydraulic injection moulding machine with a hydraulic closing unit of the Battenfeld HM800/210 type was employed. The maximum closing force is 800 kN, the screw diameter is 25 mm. A mould with a conically tapering, rectangular core was used at the test mould. For determination of the demoulding force, a load cell with a maximum measuring range of 2 kN was attached to the ejector rod. The moulding composition was predried at about 225° C. for about 4 hours. Significantly improved demoulding was observed with the thermoplastic composition according to the invention compared with an additive-free moulding composition.

### Example 11

### Preparation of a Detergent

0.2 wt. % of zinc ricinoleate (Tego®Sorb Cone 50 from Goldschmidt), 1 wt. % of sodium citrate, 0.1 wt. % of the ester obtained in Example 3, as a defoamer, 1 wt. % of boric acid, 7.5 wt. % of glycerol, 1 wt. % of ethanol, 4 wt. % of C₁₂-C₁₆-alkyl glycoside, 8 wt. % of soap, 8 wt. % of C₁₂-C₁₄-fatty alcohol+1.3 EO sulphate sodium salt, 1 wt. % of Acusol 120 (15% strength; methacrylic acid (stearyl alcohol-20 EO) ester/acrylic acid copolymer from Rohm & Haas), 0.5 wt. % of Dequest 2066, amylase, protease, and water were mixed to give a detergent.

### Example 12

### Preparation of an Adhesive

A high molecular weight diisocyanate was prepared from a polypropylene glycol of Mₙ=880 g/mol and diphenylmethane-diisocyanate in accordance with the teaching of DE-A-199 57 351, and the monomeric MDI was then removed from this until a residual monomer content of 0.1% resulted. A hot-melt adhesive was prepared from 100 parts of a polyol mixture for a standard polyurethane hot-melt adhesive (QR 6202, Henkel) having an average OH number of 32.5 and 76.5 parts of the above-mentioned high molecular weight diisocyanate. 5 wt. % of the ester prepared in Example 3 was additionally added.

### Example 13

### Preparation of a Defoamer

4.0 wt. % of paraffin having a solidification point according to DIN ISO 2207 of 45° C., a liquid content at 40° C. of about 66 wt. % and a liquid content at 60° C. of about 96 wt. %, 1.2 wt. % of bisamide, 3 wt. % of sodium carbonate, 58.7 wt. % of sodium sulphate, 21.4 wt. % of sodium silicate, 2.1 wt. % of cellulose ether, 4.8 wt. % of the ester obtained in Example 3 and water are mixed to form an aqueous slurry, which was spray dried with superheated steam by the process of the European patent specification EP 625 922.

### Example 14

### Preparation of a Defoamer II

1.2 wt. % of bisamide, 3 wt. % of sodium carbonate, 58.7 wt % of sodium sulphate, 21.4 wt. % of sodium silicate, 2.1 wt. % of cellulose ether, 8.8 wt. % of the ester obtained in Example 3 and water are mixed to form an aqueous slurry, which was spray dried with superheated steam by the process of the European patent specification EP-A-0 625 922.

### Example 15

### Preparation of a Textile Auxiliary

5 g of the polymer emulsion prepared according to Example 1b of DE-A-39 39 549 were added to 995 g of a textile lubricant comprising 84.5 wt. % of i-butyl stearate, 5 wt. % of oleyl/cetyl alcohol 5 mol EO, 2.2 wt. % of coconut fatty acid monoethanolamide 4 mol EO, 0.8 wt. % of oleic acid, 6 wt. % of secondary fatty alcohol 7 mol EO (Tergitol 1557, manufacturer: Union Carbide) and 1.5 wt. % of water at 20° C., while stirring (maximum stirring speed of an overhead stirrer with a propeller stirrer). After 30 seconds, the polymer emulsion had become uniformly distributed and a clear solution was formed. Thereafter, the stirring speed was reduced as far as possible and the textile lubricant was heated to 60° C. to accelerate dissolving of the polymer particles.

### Example 16

### Preparation of a Lacquer

736 g of demineralized water, 4 g of a 70 wt. % solution of the sebacic acid dioctyl ester prepared in Example 7 in C₁₂H₂₆ (isomer mixture), 10 g of sodium nitrobenzenesulphonate, 5 g of the tetrasodium salt of ethylenediaminetetraacetic acid, 100 g of urea, 25 g of sodium bicarbonate, 100 g of D-I.1 and 20 g of Fluorescent Brightener C.I. 230 were initially introduced into a mixing vessel. 5 g of the oleic acid n-octyl ester obtained in Example 3 were added as a defoamer and the mixture was stirred with a high-speed stirrer at 2,000 rpm for 60 seconds.

### Example 17

### Preparation of a Cosmetic Formulation

O/W emulsions were prepared, the oil phases of which had the following composition:
5.0 g of the compounds characterized by the formula (I) in EP-A-1 485 061, in which R′ represents methyl and R in each case represents a butyloctanoyl radical (C₁₂),
5.0 g of the emulsifier dioctyl ether (Cetiol OE, Cognis),
0.6 g of the emulsifier cetyl stearyl alcohol+20 EO (Eumulgin B2, Cognis),
0.1 g of creatine.

5 wt. % of the ester obtained in Example 3 was added to the composition obtained in this way.

### Example 18

### Preparation of a Drilling Mud

A conventional lime mud was prepared from 7.6 g of prehydrated bentonite, 1.15 g of ferrochrome lignosulphonate, 2.3 g of slaked lime, 0.38 g of starch and 0.76 g of NaOH. 5 wt. % of the ester obtained in Example 3 was additionally added to this lime mud.

### Example 19

### Preparation of a Soil Compacting Agent

To a commercially available polyvinyl acetate dispersion (approx. 50 wt. % of polyvinyl acetate, obtainable from Henkel KGaA, Düsseldorf), 10 wt. %, based on the aqueous dispersion, of
a) oleic acid triglyceride,
b) oleic acid n-octyl ester from Example 3, or
b) sebacic acid di(n-butyl ester) from Example 45
   is added.

As experiment d), a film was obtained from the untreated dispersion, which serves as a reference.

Films with an average thickness of (400±30) μm are produced from these dispersions a) to c) by dipcoating on rotating Teflon discs of 10 cm diameter.

These films are evaluated with respect to flexibility, water-solubility and cohesion. After 21 days, they were tested for stability and homogeneity.

In comparison with the reference experiment d), all three dispersions a) to c) show a significantly improved flexibility of the films after three weeks. Films a) to c) moreover show an extremely high cohesion compared with d) immediately after film formation and in particular also after three weeks, so that they tear only when a high force is applied.

Field experiments in which aa) layers of sand, bb) potting compost and cc) sandy loess were treated with dispersions a) to d) and exposed to weathering in the Düsseldorf, Germany region for 3 weeks proved to be highly compacted in the case of the samples treated with dispersions a) to c) compared with the sample untreated as a reference.

### Example 20

### Preparation in a Pilot Plant

The process according to Example 1 (preparation of pentaerythritol dioleate) was carried out on a 1.000-fold scale in a pilot plant. After the reaction, the ester A mixture prepared in this way was cooled to 80° C. and then mixed with a mixture of 25 kg of kieselguhr (Celite 535, Brenntag N.V., Deerlijk, Belgium) and 5 kg of active charcoal (Norit SA-2, Norit Deutschland GmbH, Kempen, Germany), with vigorous stirring. The mixture obtained in this way was led and filtered continuously over a filter press (23 chambers, 40 mm chamber depth, filter surface 13 m³, pressing volume 250 l, delivery 3 m³/h) with a circulating pump for 1 hour. A pale yellowish, clear liquid was obtained. The acid number was 1.2.

### LIST OF REFERENCE SYMBOLS ON FIG. 1

| | |
|---|---|
| Reaction region | 110 |
| Reactor | 111 |
| Heating jacket | 112 |
| Baffle | 113 |
| Stirrer shaft | 211 |
| Stirrer blade | 212 |
| Transmission | 213 |
| Motor | 214 |
| Working up unit | 311 |
| External heat exchanger | 411 |
| Feed line | 511 |
| Educt reservoir | 512 |
| Delivery pump | 911 |
| Distributing device | 912 |
| Circulation line | 921 |
| Return line, flange | 922 |
| Feed of process components | 932 |
| Vapours line | 941 |
| Heat exchanger | 942 |
| Pressure reducing unit | 945 |
| Separator with interfacial layer regulator | 946 |
| Receiver | 947 |
| Return line | 948 |
| Filling level line | F |

### LIST OF REFERENCE SYMBOLS ON FIG. 2

### Object 311

| | |
|---|---|
| Reactor | 110 |
| Working up container | 312 |
| Mixing device | 313 |
| Heating jacket | 314 |
| Delivery pump | 315 |
| Transmission | 316 |
| Motor | 317 |
| Feed | 318 |
| Delivery line | 319 |
| Distributing device | 320 |
| Return line | 321 |
| Distributing device | 322 |
| Filter press | 331 |
| Holding unit | 611 |

### LIST OF REFERENCE SYMBOLS ON FIG. 3

### Object 331

| | |
|---|---|
| Feed | 332 |
| Front plate | 333 |
| Filter chamber | 334 |
| Recipient | 335 |
| Outlet | 336 |
| Ram | 337 |
| Motor with transmission | 338 |

### LIST OF REFERENCE SYMBOLS ON FIG. 4

### Object 334

| | |
|---|---|
| Filter frame | 341 |
| Filter material | 342 |
| Filter surface | 343 |
| Filter cake | 344 |

1. A process for the preparation of an ester, at least based on
a. at least one alcohol component,
b. at least one carboxylic acid component, and
c. optionally further additives,
as process components, comprising, in a reactor, the process steps:
i. provision of the process components,
ii. reaction of the process components to give an ester A,
iii. after-treatment of the ester A,
wherein the ester A is transferred into a working up container and combined there with
aa. at least one active component which is introduced into the ester A as a particulate solid, and
bb. optionally further auxiliary substances
cc. to give a mixture, before
dd. this mixture is divided into a solid and a liquid phase, the ester being obtained as the liquid phase.

2. The process of claim 1, wherein less than 50 mol-% of unreacted carboxylic acid is neutralized with a basic compound before ester A is combined with the at least one active component.

3. The process according to claim 1, wherein from 0.01 to 20 parts of the active component to 100 parts of process components are introduced into the ester A.

4. The process according to claim 1, wherein at least 70 wt. % of the particles, based on the total weight of the active component, have a particle size in a range of from 8 μm to 0.1 mm.

5. The process according to claim 1, wherein the active component has a fine dust content of less than 30 wt. %, based on the total weight of the active component.

6. The process according to claim 1, wherein the active component has a BET surface area of from 50 to 1,500 m²/g.

7. The process according to claim 1, wherein the active component is chosen from the group consisting of: inorganic silicon-oxygen compound, active charcoal, kieselguhr, or a combination of two or more of these.

8. The process according claim 7, wherein the active charcoal has a BET surface area of from 800 m²/g to 1,100 m²/g.

9. The process according to claim 7, wherein the active charcoal consists of elemental carbon to the extent of more than 80 wt. %, based on the total amount of active charcoal.

10. The process according claim 7, wherein the inorganic silicon-oxygen compound has a BET surface area of from 150 m²/g to 204 m²/g.

11. The process according to claim 7, wherein the inorganic silicon-oxygen compound is a bentonite, in particular an acid-activated calcium bentonite.

12. The process according to claim 7, wherein a combination of at least one active charcoal and at least an inorganic silicon-oxygen compound is employed as the active component.

13. The process according to claim 12, wherein the ratio of inorganic silicon-oxygen compound to active charcoal is from 10:1 to 1:10.

14. The process according to claim 1, wherein the active component comprises less than 5 wt. % of a liquid, based on the total amount of active component.

15. The process according to claim 1,
wherein the division of the mixture in step dd. is carried out in a separating device and
wherein at least a part of the mixture is led back in a circulation from the working up container via the separating device into the working up container.

16. The process according to claim 1, wherein the after-treatment is carried out at an after-treatment temperature of between 50° C. and 100° C.

17. The process according to claim 1, wherein the ester A is after-treated over a period of time in a range of from 30 to 240 min.

18. The process according to claim 1, wherein the circulation has a throughput in a range of from 1 to 20 m³/h and is carried out for a duration of from 10 to 240 minutes.

19. The process according to claim 1, wherein the mixture for the division has a pressure in a range of from 1 to 3 bar.

20. The process according to claim 1, wherein the division is carried out at a temperature in a range of from 60 to 100° C.

21. The process according to claim 1, wherein the separating device is configured as a filter press.

22. The process according to claim 1, wherein the solid phase has a thickness in a range of between 2 and 20 mm.

23. The process according to claim 1, wherein the carboxylic acid component is chosen from the group consisting of: nonanoic acid, i-nonanic acid, decanoic acid, i-decanoic acid, sebacic acid, palmitic acid, stearic acid, oleic acid, azelaic acid, HOOC—C₃₆H₇₂—COOH, phthalic anhydride, or a mixture of two or more of these.

24. The process according to claim 1, wherein the alcohol component is chosen from the group consisting of glycerol, oligoglycerols, pentaerythritol, pentaerythritol dimer, n-octanol, i-tridecanol, pentaerythritol, pentaerythritol dimer, n-butanol, i-butanol, n-propanol, i-propanol, 2,2-dimethylpropanol, 2-ethylhexanol, n-octanol, i-tridecanol, cetyl alcohol, stearyl alcohol, ethylene glycol, diethylene glycol, butyl glycol, dibutyl glycol, tributyl glycol, polyethylene glycol or a mixture of two or more of these.

25. The process according to claim 1, wherein at least one catalyst chosen from the group consisting of proton donor or electron donor, or both, is employed as an additive.

26. The process according to claim 1, wherein the ester A has between 1 and 6 ester groups.

27. A device comprising as device units connected by fluid-conducting means
α) at least one educt reservoir,
β) a reactor with a mixing device,
γ) a working up unit,
wherein the working up unit comprises, connected by fluid-conducting means:
αα) a working up container
ββ) a delivery pump and
γγ) a separating device, and
a filter press which has two or more filter chambers which comprise at least one filter material, the filter material having a permeability to air of from 5 to 20 l·m⁻²·s⁻¹ and a weight per unit area of from 500 to 700 g/m², is employed as the separating device.

28. The device according to claim 27, wherein a filter surface is arranged on each filter material, the filter surface being characterized by at least one of the following features:
FP1) a weight per unit area of 65-75 g/m²,
FP2) a filtration speed of 20″-30″ according to DIN 53137,
FP3) a thickness of 24-30 mm,
FP4) a bursting pressure of 2.5-3.5 kp.

29. The device according to claim 27, wherein a filter cake forms in at least one filter chamber, this filter cake having a height (h) in a range of from 2 to 10 mm.

30. The device according to one of claim 27, wherein an active component is present in the working up unit.

31. The device according to claim 27, wherein the active component in the working up unit has a BET surface area in a range of from 50 to 1,500 m²/g.

32. The device according to claim 27, wherein the active component is chosen from the group consisting of: silica gel, kieselguhr or active charcoal, or a combination of two or more of these.

33. A process for the preparation of an ester, wherein a device according to claim 27 is employed.

34. A process for the preparation of a thermoplastic composition comprising the components
a1) a thermoplastic polymer,
b1) an additive, and
c1) optionally further additives,
comprising the process steps:
i) provision of a thermoplastic polymer or of a precursor of a thermoplastic polymer or both,
ii) provision of an additive comprising an ester which has been prepared by a process according to claim 1,
iii) optionally provision of further additives,
iv) mixing of components i), ii) and optionally iii).

35. The process according to claim 34, wherein the mixing is carried out in accordance with at least one of the following measures:
M1) at or above the glass transition temperature of the thermoplastic polymer,
M2) wherein the additive is more liquid than the thermoplastic polymer,
or
M3) wherein at least a part of the additive is added to the precursor of the thermoplastic polymer.

36. The process according to claim 34, wherein the thermoplastic polymer is based on polyesters to the extent of more than 90 wt. %.

37. The process according to claim 34, wherein the polyester is a polymeric ester of a polycarboxylic acid and a polyol or a polymeric ester based on a hydroxycarboxylic acid.

38. The process according to claim 34, wherein components a1) to c1) are mixed with one another in relative amounts such that the thermoplastic composition obtained by mixing components a1) to c1) contains
a11) at least 40 to 99.99 wt. % of the thermoplastic polymer,
b11) 0.01 to 60 wt. % of the additive and
c11) 0 to 20 wt. % of the further additives,
in each case based on the total weight of the thermoplastic composition, wherein the sum of components a11) to c11) is 100 wt. %.

39. A process for the production of a shaped article based on a thermoplastic composition, comprising the process steps:
I) provision of thermoplastic composition obtainable according to claim 32,
II) heating of the thermoplastic composition to the glass transition temperature or to a temperature above the glass transition temperature of the thermoplastic polymer,
III) production of a shaped article from the heated thermoplastic composition prepared in process step II).

40. The process according to claim 39, wherein in a further process step IV) at least a part region of the shaped article obtained in process step III) is reduced in its mass cross-section compared with process step III).

41. The process according to claim 40, wherein the reduction in cross-section is carried out by applying a gas pressure.

42. The process according to claim 39, wherein the shaped article is chosen from a group consisting of: a container, a film, a fibre or at least two of these.

43. A process for the production of a packed product, comprising the process steps:
a3) provision of a shaped article, obtainable according to claim 39, and a product;
b3) at least partial surrounding of the product with the shaped article.

44. A process for the production of an at least partly coated object, comprising the process steps:
a4) provision
of a coating composition comprising at least 10 wt. %, based on the coating composition, of
an ester according to claim 1, or
a thermoplastic composition obtainable according to claim 34,
or both;
and
a solid substrate;
b4) mixing of the coating composition and the substrate, wherein the coating composition is at least partly liquid.

45. A further processing product comprising an ester obtainable according to claim 1, as an additive, and at least one functional component chosen from the group consisting of thermoplastic polymer, enzyme, curing agent of an adhesive, paraffin, oil, colouring agent, hair or skin care substance, polymer dispersion, lime mud, lubricant or emulsifier, or a combination of two or more of these.

46. A use of an ester obtainable according to claim 1, as an additive in a composition which is chosen from the group consisting of: thermoplastic composition, detergent, adhesive, defoamer, lubricant formulation, lacquer, paint, cosmetic formulation, soil compacting agent, drilling mud, hydraulic oil or dispersion.

47. A use of an active charcoal which comprises elemental carbon to the extent of more than 80 wt. % and has a BET surface area in a range of from 800 to 1,100 m²/g for the purification of esters.
